(19) United States
(12) Patent Application Publication
(10) Pub. No.: US 2022/0175689 A1
(43) Pub. Date: Jun. 9, 2022

(54) NOVEL DRUG DELIVERY COMPOSITION AND PROCESS FOR BLOOD-BRAIN BARRIER CROSSING

(71) Applicant: Phosphorex, Inc., Hopkinton, MA (US)

(72) Inventors: Bin Wu, Lexington, MA (US); Nicholas Boylan, Boylston, MA (US); Zimeng Wang, Framingham, MA (US); Jieni XU, Quincy, MA (US)

(21) Appl. No.: 17/527,965

(22) Filed: Nov. 16, 2021

Related U.S. Application Data
(60) Provisional application No. 63/114,700, filed on Nov. 17, 2020.

(51) Int. Cl. A61K 9/51; A61K 45/06

(52) U.S. Cl. CPC A61K 9/5153; A61K 9/5169; B82Y 5/00; A61K 45/06; A61K 9/5192

(57) ABSTRACT

This invention provides polymeric nanoparticles presenting non-conjugated BBB-crossing ligands on their surfaces, compositions and methods of use thereof, as well as non-conjugation methods to produce nanoparticles having BBB-crossing agents on their surfaces.

### RELATED APPLICATION

This application claims the benefit of U.S. Provisional Application No. 63/114,700, filed on Nov. 17, 2020. The entire teachings of the above application are incorporated herein by reference.

### BACKGROUND OF THE INVENTION

The blood-brain barrier (BBB) formed by the brain microvascular system is a highly selective semipermeable border of endothelial cells that prevents solutes in the circulating blood from non-selectively crossing into the extracellular fluid of the central nervous system (CNS) of most vertebrates including amphibians, reptiles, birds, and mammals. The BBB is formed by endothelial cells of the capillary wall, astrocyte end-feet ensheathing the capillary, and pericytes embedded in the capillary basement membrane. The BBB protects the brain from foreign substances in the blood that may damage the brain and keep a constant brain environment. The BBB only allows the passage of some molecules by passive diffusion, as well as the selective transport of various nutrients, ions, organic anions, and macromolecules such as glucose, water and amino acids that are crucial to neural function.

The BBB is a major obstacle for delivering drugs to treat brain tumors and neurodegenerative diseases such as Alzheimer's and Parkinson's diseases. While small-molecule hydrophobic drugs can pass through the BBB by passive diffusion, most existing drug products and drug candidates cannot be delivered into the brain because of their high hydrophilicity.

Numerous approaches have been studied in laboratories and clinic to overcome the BBB obstacle. For example, administration methods such as intracranial, intrathecal, or intraventricular injections, along with chemical BBB disruption, may facilitate the delivery of some drugs to the brain, but these approaches are invasive and incompatible with repeat dosing regimens. Hyperosmolar solutions, microbubbles and focused ultrasound (FUS) have also been employed to temporarily disrupt the BBB and increase permeability. However, these methods may cause various issues such as the leakage of membrane proteins, the entry of toxins or pathogens into the CNS, the release of cytokines, and imbalance of ions and transmitters, which lead to neuronal dysfunction, inflammation and/or degeneration.

Recently much effort has been made toward developing receptor-mediated transcytosis (RMT) as a noninvasive strategy for enhancing delivery of biotherapeutics (Kariolis et al., Sci. Transl. Med. 12, eaay1359 2020; Y. J. Yu, R. J. Watts, Developing therapeutic antibodies for neurodegenerative disease, Neurotherapeutics 10, 459-472, 2013; A. R. Jones, E. V. Shusta, Blood-brain barrier transport of therapeutics via receptor-mediation, Pharm. Res. 24, 1759-1771, 2007). RMT is an endogenous process wherein biomolecules such as transferrin, insulin, peptides, and lipids bind to cognate receptors on brain endothelial cells and are subsequently transported across the BBB. Protein therapeutics engineered to bind these brain endothelial cell-enriched receptors can similarly exploit RMT as a means of CNS delivery. However, such RMT-based delivery strategies have relied upon antibody or antibody fragment binding to engage brain endothelial cell receptors, and development and production of antibodies can be costly. In addition, antibodies are prone to hydrolysis and can cause immunogenicity.

For over two decades, various types of nanoparticles (NPs) have been used in research laboratories and clinical studies to deliver drug molecules across the BBB. Nanoparticle-mediated BBB crossing offers many advantages including non-invasiveness, low cost, good biodegradability and long-term stability, ease of synthesis, high targeting efficiency, and high controllability to load and release drugs across the BBB.

Nanoparticles are solid colloidal particles with a size range of 1-1,000 nm. Typically, a drug is loaded into a nanoparticle and a BBB-crossing agent is attached on the surface of the nanoparticle.

Nanoparticles that have been used in facilitating drug delivery to the brain include polymer nanoparticles, liposome, solid lipid nanoparticles, metal and metal oxide nanoparticles, micelles, silica nanoparticles, and carbon quantum dots.

Agents that have been found in research and development to assist BBB crossing include transferrin, insulin, cell-penetrating peptides, glutathione, cationic proteins, albumin, chitosan, aptamers, and surfactants like polysorbate 80 and Poloxamer 188. Transport mechanisms across BBB include receptor-mediated endocytosis, adsorption-mediated endocytosis, and carrier-mediated transport. After surface coating with specific compounds such as polysorbate 80, the BBB penetration efficiency of NPs could be significantly enhanced via active transport mechanisms other than simple passive diffusion.

Two methods, chemical conjugation and physical coating, are commonly used to attach the BBB-crossing agent to the surface of nanoparticles for delivery of drugs into the brain.

Chemical conjugation involves chemically reacting the ligand with a reactive group on the nanoparticle. This process allows for the formation of covalent bonding between the ligand and the nanoparticle. However, this method suffers from low conjugation efficiency due to steric hinderance and insufficient reactivity. In addition, the conjugation process can generate non-biocompatible side products which may be harmful to the patient.

In a physical coating process, pre-formed nanoparticles are mixed and incubated with a solution containing the ligand to be coated resulting in the formation of a loose coating of the ligand on the nanoparticle surface. Since no covalent bonding is formed during the coating process, the coated ligand layer may be detached from the nanoparticle surface during necessary purification steps. Therefore, the coating is not durable and can be easily washed away from the nanoparticle surface.

Therefore, there is an unmet need for novel methods to attach BBB-crossing ligands to the surface of nanoparticles for delivering drugs into the brain.

### SUMMARY OF THE INVENTION

This invention provides a pharmaceutical composition comprising nanoparticles for delivering active ingredients to cross the blood-brain barrier (BBB) into the brain, said nanoparticles comprising active ingredients incorporated in the nanoparticles and BBB-crossing ligands on the surface of the nanoparticles, wherein said BBB-crossing ligands are tightly attached on the surface of said nanoparticles without covalent bonding. This invention also provides a process of making said nanoparticulate composition and the method of using such composition to treat certain diseases in mammals. Specifically, this invention provides a process without the need for chemical conjugation for creating a durable coating of certain BBB-crossing ligands on the surface of drug-loaded nanoparticles.

The invention includes a composition comprising polymeric nanoparticles presenting a BBB-crossing ligand on their surfaces, wherein each nanoparticle comprises a biodegradable polymer and a BBB-crossing ligand, wherein the BBB-crossing ligand is not conjugated to the surface of the nanoparticles. The biodegradable polymer is preferably a pharmaceutically acceptable biodegradable polymer. In certain aspects, the biodegradable polymer can be selected from the group consisting of polylactide (PLA), poly(lactide-co-glycolide) (PLGA), copolymers of ethylene glycol and lactide/glycolide (PEG-PLGA), copolymers of ethylene glycol and lactide (PEG-PLA), copolymers of ethylene glycol and glycolide (PEG-PGA), poly(ethylene glycol) (PEG), polycaprolactone (PCL), polyanhydrides (PANH), poly(ortho esters), polycyanoacrylates, poly(hydroxyalkanoate)s (PHAs), poly(sebasic acid), polyphosphazenes, polyphosphoesters, modified poly(saccharide)s, poly(amino esters), dendrimers, chitosan, gelatin, hyluronic acid, dextran, mixtures and copolymers thereof. In certain embodiments, the biodegradable polymer is PLGA. In certain embodiments, the biodegradable polymer is poly(n-butyl cyanoacrylate). In additional aspects, the biodegradable polymer and the BBB-crossing ligand form an interpenetrating network. The nanoparticles can further comprise an active agent such as an active pharmaceutical ingredient.

The invention additionally encompasses a method for administration of an active agent to a subject in need thereof comprising administering to said subject the composition comprising nanoparticles presenting BBB-crossing ligands on their surfaces, wherein each nanoparticle comprises a biodegradable polymer and a BBB-crossing ligand, wherein the BBB-crossing ligands are not conjugated to the surface of the nanoparticles; and further wherein the nanoparticles comprise the active agent. The active agent can be an active pharmaceutical ingredient. In certain aspects, the active agent is encapsulated within the particles.

The invention further includes a method of treating a disease or disorder in a subject in need thereof comprising administering to the subject the nanoparticles described herein.

The invention includes a method for the preparation of nanoparticles presenting BBB-crossing agents on their surfaces comprising: (1) dissolving a biodegradable polymer (and optionally an active agent, such as a pharmaceutical ingredient (API), or a poorly water soluble compound) in a first solvent to form a polymer solution; (2) emulsifying the polymer solution in a solution of a second solvent to form an emulsion, wherein the first solvent is not miscible or partially miscible with the second solvent, and wherein the solution of the second solvent comprises a BBB-crossing agent, said solution of the second solvent optionally further comprising a surfactant and/or an API soluble in the second solvent; and, (3) removing the first solvent to form said nanoparticles having the BBB-crossing ligand on their surface.

The invention also provides a method for the preparation of nanoparticles presenting BBB-crossing ligands on their surfaces, said method comprising: (1) dissolving a biodegradable polymer (and optionally an active agent, an API, or a poorly water soluble compound) in a first solvent to form a polymer solution; (2) adding a first solution of a second solvent to the polymer solution to form a mixture, wherein the first solvent is not miscible or partially miscible with the second solvent, and wherein the first solution of the second solvent optionally comprises an active agent which may be the same or different from the API dissolved in the first solvent; (3) emulsifying the mixture to form a first emulsion; (4) emulsifying the first emulsion in a second solution of the second solvent to form a second emulsion, wherein the second solution of the second solvent comprises a BBB-crossing ligand, and optionally further comprises a surfactant; and, (5) removing the first solvent to form nanoparticles having the BBB-crossing ligand on their surface.

Langer and co-workers (B. Raudszus, D. Mulac, K. Langer, Int. J. Pharm. 2018, 536, 211) taught a method of conjugating ApoE to drug-loaded nanoparticles for brain delivery. Such nanoparticles showed improved uptake by endothelial cells. However, the disclosed process was very complex. It involves a vinyl sulfone-modified poly(vinyl alcohol)-derivative (VS-PVA), a specially designed steric stabilizer bearing reactive vinyl sulfone-groups for ligand coupling reactions directly to PVA-stabilized nanoparticles. The drug loaded nanoparticles were first prepared and the surface of the nanoparticles was PEGylated by reaction of the vinyl-sulfone groups of VS-PVA-PLA with the amino groups of α-amino-ω-carboxy PEG chains. Afterward, ApoE was coupled to the PEG chains by esterification.

The current invention provides a method and process for durably attaching BBB-crossing ligands on the surface of drug-loaded nanoparticles for delivering drugs to the brain without the need for chemical conjugation. Such durably attached ligands are tightly anchored on the nanoparticle surface and can sustain multiple washing cycles. More importantly, the ligands would continue to stay on the surface of the nanoparticle after the nanoparticles are administered and while circulating in the blood and eventually guide the nanoparticle to cross the BBB to enter the brain.

In order to reduce the amount of free BBB-crossing agent entering the brain, the invention further provides an optional washing step to remove free BBB-crossing agent in the supernatant of the nanoparticle suspension. The washing step can be accomplished by centrifugation, diafiltration, tangential flow filtration or other commonly used washing and separation methods.

In some embodiments, after the optional washing step described above, the concentration of the free BBB-crossing agent in the final nanoparticle suspension (prior to lyophilization) is preferably less than 1 mg/ml, more preferably less than 0.1 mg/ml, even more preferably less than 0.01 mg/ml.

Such purified nanoparticles can be further lyophilized or kept frozen for storage.

In yet other aspects, the invention is directed to nanoparticles produced by a method described herein.

Preferably, the nanoparticles comprise an active agent, such as an active pharmaceutical ingredient (an API).

Preferably, the API is encapsulated within the nanoparticles.

Alternatively or additionally, the API is covalently or ionically attached to the surface of the nanoparticles or to the biodegradable polymers. For example, the API can be covalently attached to the nanoparticle surface or to the biodegradable polymers via a hydrolysable bond that facilitates in vivo release.

Preferably, the solution of the second solvent further comprises, or is saturated with, the first solvent before the polymer solution in the first solvent is added to the first solution of the second solvent during emulsification. This may be beneficial in that the polymer in the first solvent is less likely to precipitate when added to the first solution of the second solvent for emulsification. Preferably, the first solvent is ethyl acetate, and the solution of the second solvent (e.g., water or aqueous solution) comprises about 7-8% v/v of ethyl acetate.

Preferably, said nanoparticles are based on biodegradable polymers selected from the group consisting of polylactide (PLA), poly(lactide-co-glycolide) (PLGA), copolymers of ethylene glycol and lactide/glycolide (PEG-PLGA), copolymers of ethylene glycol and lactide (PEG-PLA), copolymers of ethylene glycol and glycolide (PEG-PGA), poly(ethylene glycol) (PEG), polycaprolactone (PCL), polyanhydrides (PANH), poly(ortho esters), polycyanoacrylates, poly(hydroxyalkanoate)s (PHAs), poly(sebasic acid), polyphosphazenes, polyphosphoesters, modified poly(saccharide)s, poly(amino esters), dendrimers, chitosan, gelatin, human serum albumin (HSA), hyluronic acid, dextran, mixtures and copolymers thereof. In certain embodiments, the biodegradable polymer is PLGA. In certain embodiments, the biodegradable polymer is poly(n-butyl cyanoacrylate) (PBCA).

Optionally, the nanoparticles comprise an active agent such as a drug. In certain preferred embodiments, the particles encapsulate the active agent.

The BBB-crossing ligand or agent (the words agent and ligand are used interchangeably herein) is preferably a peptide or protein, such as a peptide having at least about 50 amino acids in length, such as at least about 100 amino acids in length. Preferred BBB-crossing ligand is selected from the group consisting of transferrin, lactoferrin, insulin, low-density lipoprotein (LDL), apolipoproteins (such as ApoE), cell-penetrating peptides (such as penetratin), anti-transferrin receptor (TfR) antibody, ligands for transport proteins (such as GLUT1 and ACST2) or BBB-binding fragments of any of the above. Typically, the BBB-crossing ligand has affinity to BBB components, such as the molecules that form tight junctions. The BBB crossing ligand can include proteins or peptides that facilitate carrier-mediated transcytosis, receptor-mediated transcytosis, and adsorptive-mediated transcytosis.

In some embodiments, polymeric surfactants, such as polyvinyl alcohol (PVA), polyvinylpyrrolidone (PVP), a polysorbate (Tween series) surfactant, a PEO-PPO-PEO poly(ethylene oxide) poly(propylene oxide) triblock copolymer (Pluronic series or Poloxamer series) surfactant, or a t-octylphenyl-polyethylene glycol (Triton X-100) surfactant or a D-α-tocopheryl polyethylene glycol succinate (TPGS), can further improve nanoparticle delivery. Preferably, the surfactant is selected from the group containing Tween polysorbate series, Poloxamer series and mixture thereof (see examples).

More than one BBB-crossing ligand (e.g. two or three) can be incorporated to the nanoparticle surface to enhance the BBB-penetrating effect. For example, pairs of BBB-crossing ligands may include, transferrin/ApoE, transferrin/Tween-80, ApoE/Tween-80, Poloxamer 188/ApoE, insulin/Tween-80, and penetratin/Poloxamer 188, etc.

In some embodiment, a “secondary” targeting ligand is also presented on the surface of the nanoparticles along with said BBB-crossing ligand. The purpose of incorporating such secondary targeting ligand is for such drug-loaded nanoparticles to be able to further target specific disease sites or cells after they enter the brain. For example, anti-HER2 or anti-VEGF or binding fragments of the antibody can be attached to nanoparticle surface along with a BBB-crossing agent to assist in cell targeting upon cross the BBB.

Such secondary targeting ligands may also be an agent that targets tissues, cells or receptors that are associated with neurological or neurodegenerative diseases.

The nanoparticles of can incorporate or encapsulate an active pharmaceutical ingredient, or API. The API can be a small molecule, a peptide, a protein or its fragment, an antibody or its fragment, an RNA, a DNA, an oligonucleotide, and an enzyme. Typically, the API is an active agent that can treat brain cancer (such as glioblastoma), neurological diseases (such as multiple sclerosis, myasthenia gravis, etc.), neurodegenerative diseases (such as Parkinson's Disease and Alzheimer's Disease) or pain.

### BRIEF DESCRIPTION OF THE DRAWINGS

FIG. 1 is a schematic for making an ASO-Cy7-conjugate.

FIG. 2 is a schematic for the preparation of an oligonucleotide-loaded nanoparticles with an agent, such as transferrin, on the surface.

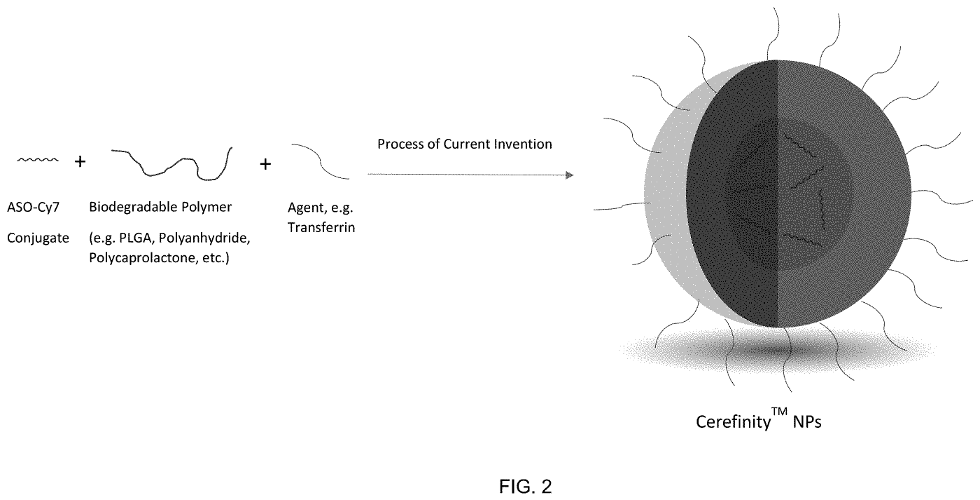

FIG. 3 is a particle size distribution graph of a typical product batch.

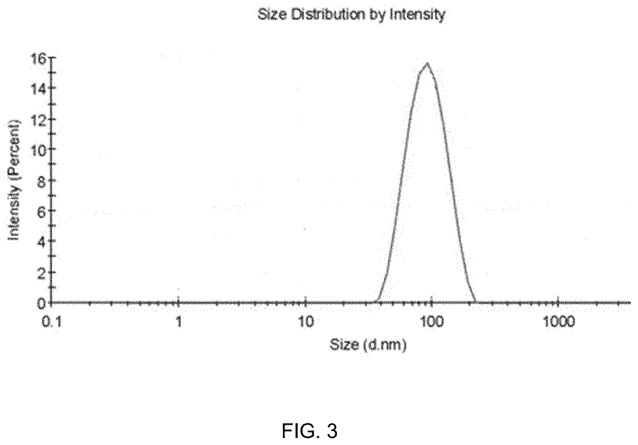

FIG. 4 is a Near IR-fluorescent imaging of brain tissue.

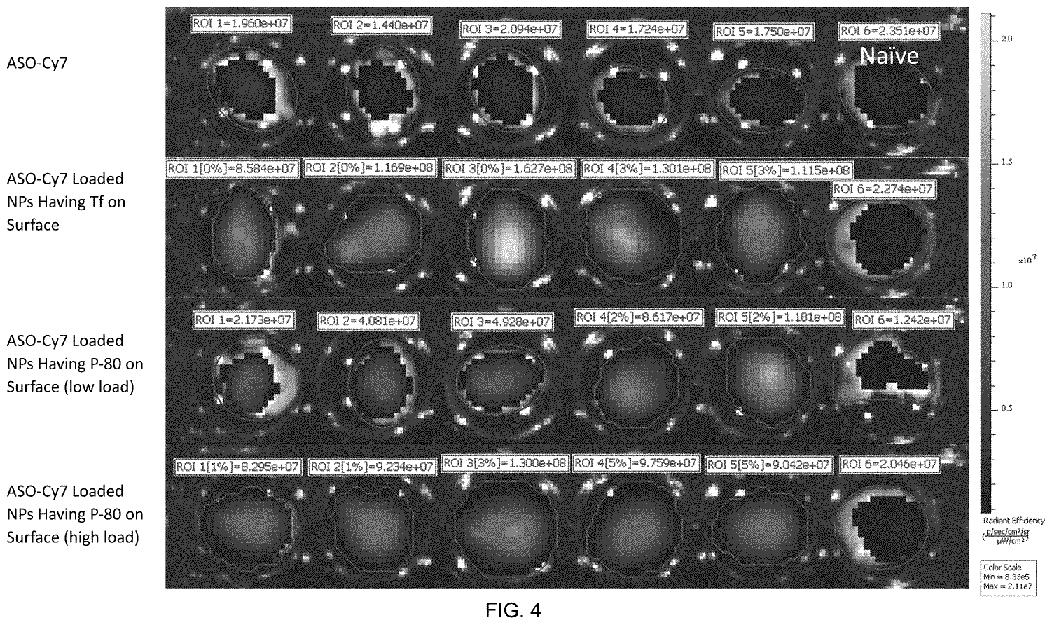

FIG. 5 is a chart showing the total radiant efficiency in brain tissue (ex vivo). Groups 1 to 4 are shown left to right. Group 1 is ASO-Cy7; Group 2 is ASO-Cy7 loaded nanoparticles (NPs) having transferrin on the surface; Group 3 is ASO-Cy7 loaded nanoparticles (NPs) having a low load of polysorbate-80 on the surface; and Group 4 is ASO-Cy7 loaded nanoparticles (NPs) having a high load of polysorbate 80 on the surface.

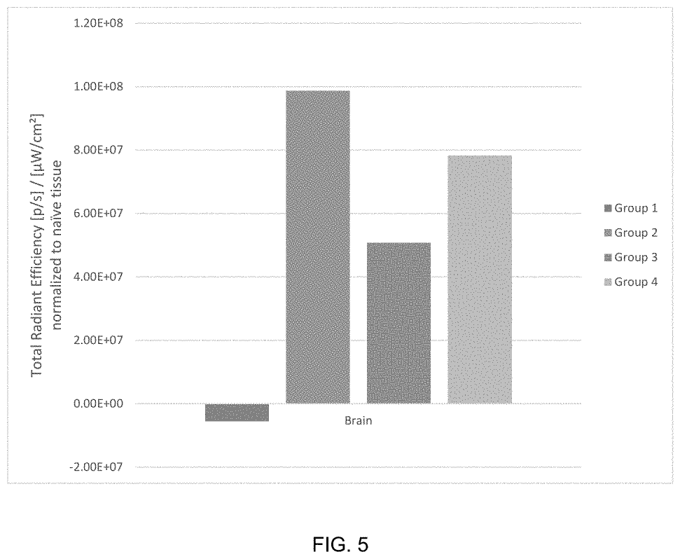

FIG. 6 is a chart showing average radiant efficiency in brain tissue for each group. Groups 1 to 4 are shown left to right. Group 1 is ASO-Cy7; Group 2 is ASO-Cy7 loaded nanoparticles (NPs) having transferrin on the surface; Group 3 is ASO-Cy7 loaded nanoparticles (NPs) having a low load of polysorbate-80 on the surface; and Group 4 is ASO-Cy7 loaded nanoparticles (NPs) having a high load of polysorbate 80 on the surface.

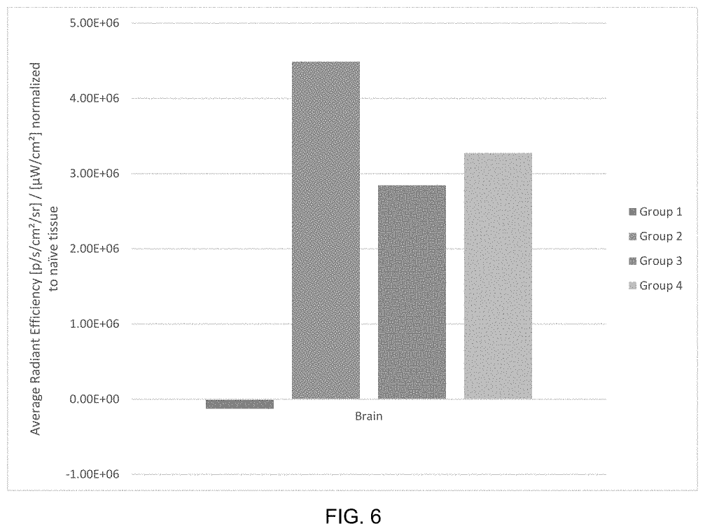

FIG. 7 is a line graph showing total radiant efficiency in brain tissue (in vivo).

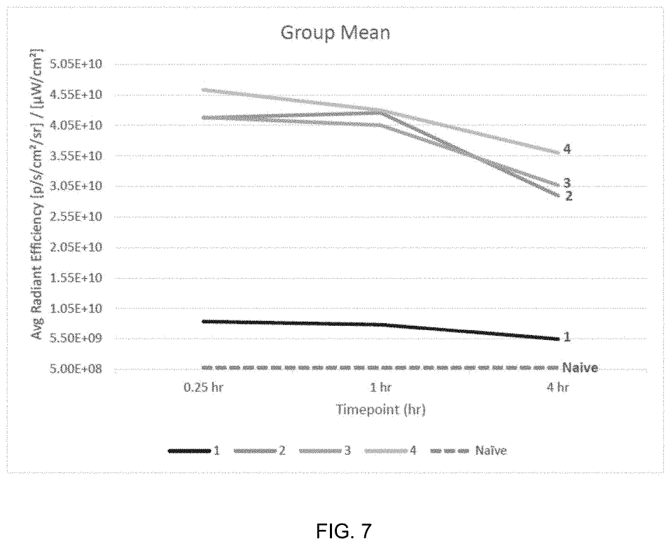

FIG. 8 is a line graph showing average radiant efficacy in brain tissue (in vivo).

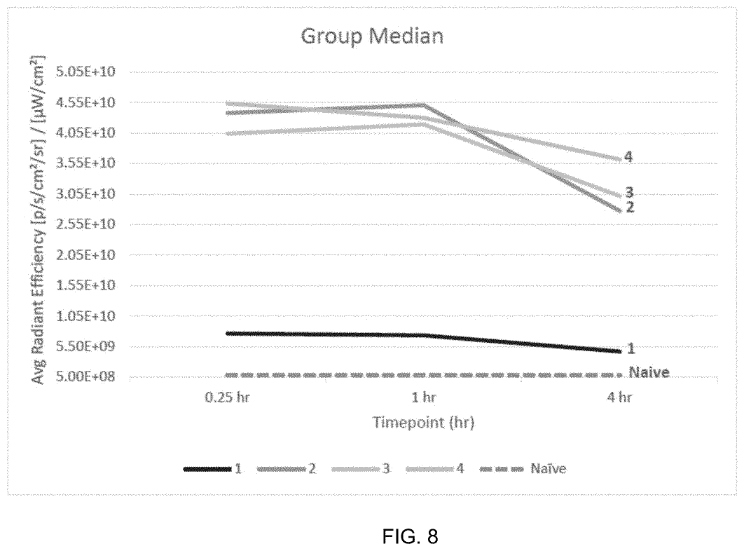

FIG. 9 are histological images of brain tissue.

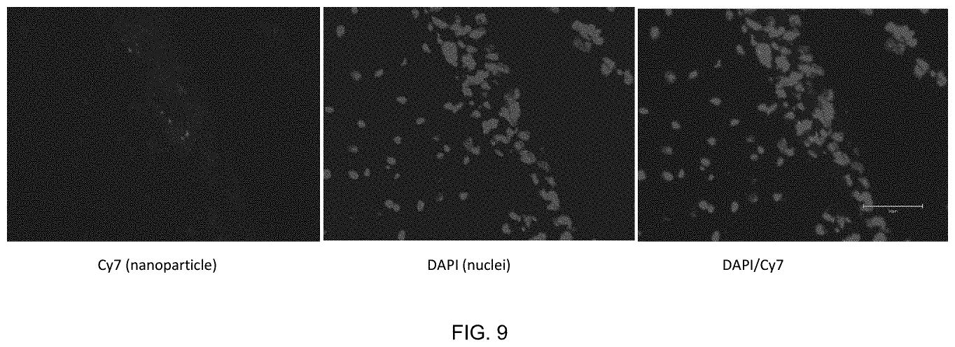

FIG. 10 are confocal microscopy slides showing colocalization of ASO-Cy7 nanoparticles having transferrin on surface with neuronal cells.

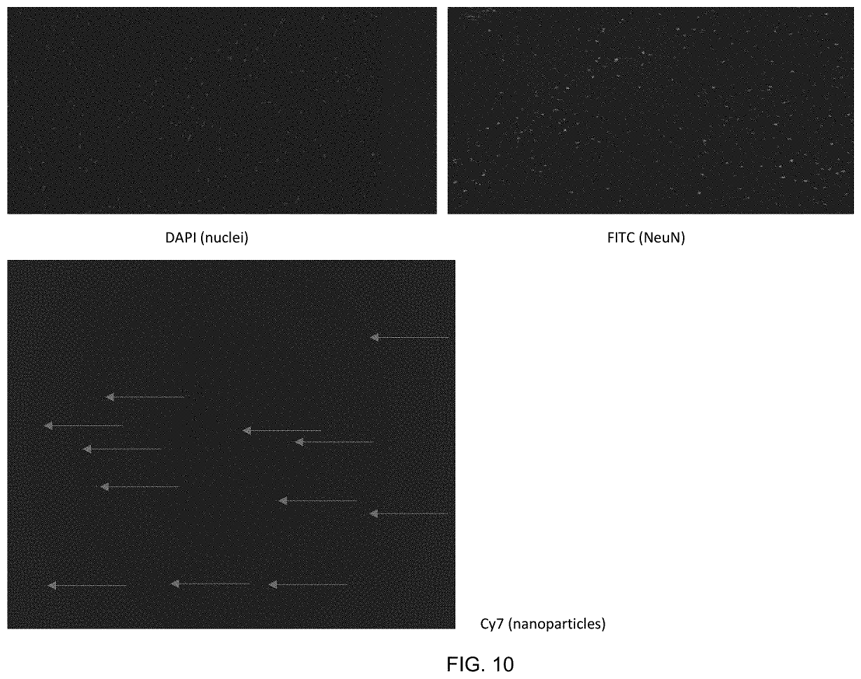

FIG. 11 are confocal microscopy slides showing colocalization of ASO-Cy7 nanoparticles having transferrin on surface with neuronal cells (continued from FIG. 10).

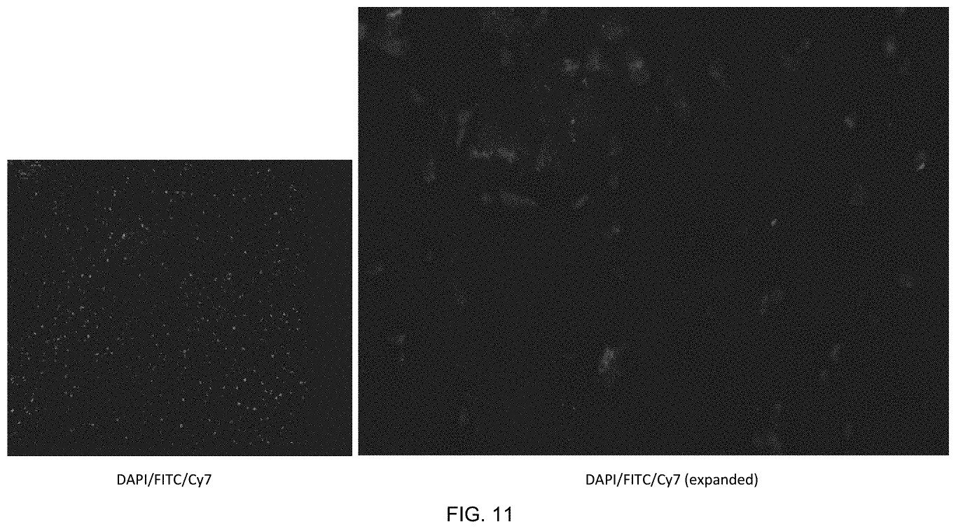

### DETAILED DESCRIPTION OF THE INVENTION

### Overview

The present invention provides particles presenting non-conjugated BBB-crossing ligands on their surfaces, compositions, and methods of use thereof as well as non-conjugation methods to produce nanoparticles having BBB-crossing ligands on their surfaces. The non-conjugation methods described herein avoid the side reactions and side-products that have been observed when using conjugation methods to attach BBB-crossing agents to the surface of nanoparticles.

The invention described herein provides pharmaceutical formulations comprising nanoparticles having BBB-crossing ligands on their surfaces (with or without agent/drug/API load), as well as processes capable of producing such pharmaceutical formulations comprising nanoparticles.

The invention includes methods for the preparation of the nanoparticles presenting BBB-crossing ligands on their surfaces, the methods comprising emulsification of a hydrophobic and/or neutral biocompatible polymer, such as PLGA or PBCA, and the BBB-crossing agent. Without being bound by any theory, it is believed that the polymer backbones intertwine or interlace while in the organic phase of emulsion. Using the methods of the invention, the BBB-crossing agent is tightly integrated into the produced nanoparticles. Thus, preferably, the BBB-crossing ligand is incorporated onto said nanoparticles and presents said ligand on the surfaces of said nanoparticles.

With the invention generally described above, specific aspects of the invention are described further in the sections below.

### Definitions

As used herein, “pharmaceutically acceptable” includes those compounds, materials, compositions, and/or dosage forms which are, within the scope of sound medical judgment, suitable for medical or veterinary use when in contact with the tissues of human beings and animals at the concentration, dosage or amount present in the product, without causing excessive toxicity, irritation, allergic response, or other problems or complications, commensurate with a reasonable benefit/risk ratio. Preferably, a pharmaceutically acceptable material (e.g., polymer, excipient, surfactant, solvent, or microparticles/nanoparticles produced therefrom) is suitable or approved for human medical use.

As used herein, “nanoparticles” are preferably roughly round, sphere, or sphere-like in shape, and are generally within the size range of, e.g., between about 1-1,000 nm, between about 10-1,000 nm, or between about 50-1,000 nm, or between about 100-500 nm, as measured by dynamic light scattering, for example. The subject nanoparticles may also include particles that are less likely to clump in vivo.

Particle size and size distribution can be measured by a dynamic light scattering instrument, e.g., a Malvern Zetasizer. Alternative techniques include, for example, sedimentation field flow fractionation, photon correlation spectroscopy, light scattering, dynamic light scattering, light diffraction, and disk centrifugation. The terms “microparticle” and “nanoparticle” are not intended to convey any specific shape limitation. Such particles include, but are not limited to, those having a generally polyhedral or spherical geometry. Preferred particles are characterized by a spherical geometry typically produced by emulsion-based encapsulation processes. It is understood that the terms “microparticle” and “nanoparticle” are used interchangeably herein, unless accompanied by a specific description of size. For example, the term “microparticles” is intended to also embrace “nanoparticles” as if stated as “microparticles and/or nanoparticles” unless the context demands otherwise.

As used herein “a” or “an” means one or more unless otherwise specified.

As used herein, “about” generally means up to +10% of the particular term being modified.

As used herein the term “encapsulates”, “encapsulated,” and the like when referring to the drug or active agent being encapsulated within the particles means that the drug or active agent is more likely found within the nanoparticle than on the surface of the nanoparticle.

As used herein, “conjugation” or “conjugated,” and the like, in the context of a BBB-crossing ligand on the surface of the nanoparticle(s) refers to the covalent association of the ligand to the nanoparticle or biodegradable polymer by formation of a covalent bond, for example, via a linker moiety or functionalization of the ligand with a reactive group capable of forming a covalent bond with a reactive group on the nanoparticle surface (e.g., a reactive group of the biodegradable polymer). Thus “not conjugated” or “non-conjugated,” and the like, in the context of BBB-crossing ligand on the surface of the nanoparticle(s) mean that the ligand is not covalently associated with the nanoparticle or biodegradable polymer by formation of a covalent bond therebetween. Without wishing to be bound by theory, it is believed that the biodegradable polymer, such as PLGA, and the ligand form an interpenetrating network presenting the ligand on the surface of the formed nanoparticles.

As used herein, the term “subject” is used to mean an animal, preferably a mammal, including a human or non-human. The terms “patient” and “subject” may be used herein interchangeably.

“Treatment” or “therapy” of a subject refers to any type of intervention or process performed on, or the administration of an active agent to, the subject with the objective of reversing, alleviating, ameliorating, inhibiting, slowing own or preventing the onset, progression, development, severity or recurrence of a symptom, complication, condition or biochemical indicia associated with a disease. As used herein, “treatment” (and grammatical variations thereof such as “treat” or “treating”) includes to clinical intervention to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, combinations of the invention are used to delay development of a disease or to slow the progression of a disease.

### Biodegradable Polymer

A biodegradable polymer is a polymer that can be metabolized or decomposed by a living thing. In certain aspects, the biodegradable polymer is decomposed or is metabolized without causing substantial toxic effects. The biodegradable polymer of the current invention can be selected from the group consisting of: polylactide (PLA), poly(lactide-co-glycolide) (PLGA), copolymers of ethylene glycol and lactide/glycolide (PEG-PLGA), copolymers of ethylene glycol and lactide (PEG-PLA), copolymers of ethylene glycol and glycolide (PEG-PGA), poly(ethylene glycol) (PEG), polycaprolactone (PCL), polyanhydrides (PANH), poly(ortho esters), polycyanoacrylates, poly(hydroxyalkanoate)s (PHAs), poly(sebasic acid), polyphosphazenes, polyphosphoesters, modified poly(saccharide)s, poly(amino esters), dendrimers, chitosan, gelatin, human serum albumin (HSA), hyluronic acid, dextran, mixtures and copolymers thereof. PLGA and polycyanoacrylates are preferred biodegradable polymers in this invention.

### PLGA

PLGA is typically prepared by ring-opening polymerization of lactide and glycolide. In this reaction, Stannous octoate is usually used as the catalyst, although other catalysts may also be used. An initiator, such as an alcohol, is often used to initiate the polymerization reaction. If no initiator is intentionally added, trace amount of polar compound containing an active proton, such as alcohol and water, may serve as the initiator. Polymerization usually results in a PLGA polymer with a carboxyl group at the chain terminal, as illustrated below:

R—OH+L(lactide monomer)+G(glycolide monomer)=PLGA-COOH

Therefore, each PLGA and/or PLA polymer molecule is typically linear, and typically contains a single COOH group at the chain terminal.

The instant invention provides various methods or combinations thereof for producing PLGA/PLA nanoparticles with a BBB-crossing agent on their surface. Such nanoparticles are particularly useful, for example, to treat certain diseases in the CNS (such as brain tumor, neurodegenerative diseases) and for delivering an active agent into the brain.

Preferably, the average molecular weight of the pharmaceutically acceptable polymer PLGA is within a desired range.

The low end of the range is preferably no less than about 100, 200, 300, 400, 500, 600, 700, 800, 900, 1000, 1200, 1500, 2000, 2500, or 3000 Da. The desired range has a low end of any of the above values.

The high end of the range is preferably no more than 50,000, 40,000, 35,000, 30,000, 25,000, 20,000, 15,000, 10,000, 7,500, or 5,000 Da. The desired range has a high end of any of the above values.

For instance, the desired range may be from about 500 to about 50,000 Da, or from about 1,000 to about 30,000 Da.

Preferably, the PLGA has an average molecular weight of from about 500 to about 1,000,000 Da, preferably from about 1,000 to about 50,000 Da.

For PLGA, average molecular weight can be expressed in other physical properties such as inherent viscosity. Inherent Viscosity (IV) is a viscometric method for measuring molecular size. IV is based on the flow time of a polymer solution through a narrow capillary relative to the flow time of the pure solvent through the capillary. For certainty measures in the instant application, the solvent used is typically chloroform, and the polymer concentration is about 0.5% (w/v). The temperature at which the viscosity is measured is about 30° C. The units of IV are typically reported in deciliters per gram (dL/g). Thus, for example, PLGA used in the instant invention may have an inherent viscosity of from about 0.01 to about 20 dL/g, or from about 0.05 to about 2.0 dL/g.

The composition and biodegradability of the subject PLGA polymer is partly determined by the molar ratio of lactide (L) to glycolide (G) unit in the polymer, or L/G ratio. The L/G ratio of the PLGA polymer in the present invention can be from 100/0 to 0/100. As used herein, an L/G ratio of “100/0” refers to polylactide or PLA, and an L/G ratio of “0/100” refers to polyglycolide, or PGA. Preferably, the L/G ratio for the PLGA polymer is from about 100/0 to 0/100, or about 95/5 to 5/95, more preferably from about 85/15 to 15/85. The most preferable L/G ratio in the present invention is about 50/50.

Other polymers can be mixed with the PLGA polymer in the preparation of the PLGA nanoparticles. For example, polyethylene glycol, or PEG, is often added to the PLGA for enhanced performance. PEGylated nanoparticles are useful because they often have increased circulation time in human or animal bodies.

Preferably, copolymers of PEG and PLGA can also be used.

The microparticles and nanoparticles prepared from the PEG and PLGA mixture or PEG and PLGA copolymer are referred to as PEGylated PLGA microparticles and nanoparticles.

Such “PEGylation” process can also be done after nanoparticles are formed. In this case, PEG polymers or other polymers containing PEG units are coated via physical absorption onto the PLGA nanoparticles.

The PEG units can also be attached to the surface of PLGA nanoparticles via covalent bonds. Such process is often referred to as “conjugation.” In a conjugation process, a reactive entity containing PEG units react with certain functional groups on the surface of the microparticles and nanoparticles to form chemical bonds.

Thus, preferably, the pharmaceutically acceptable polymer is PLGA, and the nanoparticles are PEGylated. The nanoparticles may be PEGylated by mixing polyethylene glycol (PEG) or PEG-containing entity during the preparation of the nanoparticles. The nanoparticles may also be PEGylated by using copolymers of PEG and PLGA. The nanoparticles can further be PEGylated by physically absorbing PEG polymers or polymers containing PEG units onto the PLGA nanoparticles. The nanoparticles may additionally be PEGylated by conjugating PEG units to the surface of the PLGA nanoparticles via covalent bonds.

Preferably, the biodegradable polymer has an average molecular weight of from about 500 to about 1,000,000 Da, preferably from about 1,000 to about 200,000 Da.

Preferably, the biodegradable polymer is PLGA and has an L/G ratio of from about 100/0 to 0/100, about 95/5 to 5/95, about 85/15 to 15/85, and about 50/50.

### Polycyanoacrylates

Polycyanoacrylates, also referred to as poly(alkyl cyanoacrylates), are polymers of cyanoacrylates having the structure shown below.

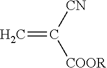

Where R is an alkyl group and can be, for example, methyl, ethyl, isopropyl, n-butyl, octyl, etc.

Poly(n-butyl cyanoacrylate), PBCA, is especially useful and therefore is preferred in the current invention.

Polycyanoacrylates are typically prepared by free-radical polymerization of the monomer, cyanoacrylates. A free-radical initiator (such as benzoyl peroxide, azobisisobutyronitrile, and potassium persulfate) initiates the polymerization. Such polymerization can be carried out in bulk, solution, emulsion, suspension or dispersion. Although nanoparticles of polycyanoacrylates can be directly prepared by emulsion polymerization of cyanoacrylates. It is preferred in the current invention that the nanoparticles of polycyanoacrylates be prepared by first dissolving polycyanonacrylates in a solvent followed by emulsifying the polymer solution in an aqueous solution and evaporating the solvent (see examples).

### Active Agent

The nanoparticles described can further comprise an active agent. The composition can comprise an API, and the API can be covalently or ionically attached to the surface of the nanoparticles via covalent bonds, such as a bond formed between an amide group of a protein and a carboxyl group on the surface of the nanoparticle. The API can also be encapsulated within the nanoparticles. The amount of the API can be about 0.001 to about 50% (w/w) of the microparticle or nanoparticle, or about 0.005 to about 25%, about 0.01 to about 10%, about 0.02 to about 5%, about 0.05 to about 3%, about 0.1 to about 5%, or about 0.2 to about 5% (w/w) of the nanoparticle.

In certain aspects, the active agent is advantageously a drug (also referred to herein as an active pharmaceutical ingredient, or API). However, active agents that are non-therapeutic can also be included as part of the nanoparticles according to the methods. For example, agents useful in diagnostics, agriculture, cosmetics, personal products, home products, industrial chemicals, dyes, fluorescing agents, coloring agents, imaging agents and the like can be included. Preferred active ingredients include small molecules and macromolecules. For example, biomolecules, such as peptides, peptidomimetics, oligonucleotides, nucleic acid molecules and mimics thereof, such as DNA, RNA, PNA, siRNA, microRNA, antisense, proteins, antibodies and antigen binding fragments thereof, enzymes, hormones, growth factors, antigens, neoantigens, saccharides, oligosaccharides, polysaccharides, and a combination thereof. The composition can be free from other active pharmaceutical ingredients or API, such as attached peptide or antigenic moieties. It is understood that an API can be substituted with non-therapeutic compounds, such as diagnostic, agricultural, or chemical agents. Therefore, in each instance where the term API is used, it shall be understood that the term “active agent,” including diagnostic, agricultural or chemical agents can be used in lieu thereof. The term “API” and “drug” are used interchangeably herein.

The API can be water-soluble or have relatively poor water-solubility. For example, a poorly water-soluble API may be dissolved in the same first solvent used to dissolve the biodegradable polymer, or be dissolved in a suitable solvent (that may be the same or different from the first solvent) to form an API solution, before the API solution is mixed with the first solvent comprising the biodegradable polymer, such that the API and polymer both remain in the resulting solution. A water-soluble API may be first dissolved in its own solvent (that may be the same or different from the 2^{nd} solvent) to form an API solution, before the API solution is added to the second solvent.

An API or active agent can include a wide variety of different compounds, including chemical compounds and mixtures of chemical compounds, e.g., small organic or inorganic molecules; saccharins; oligosaccharides; polysaccharides; biological macromolecules, e.g., peptides, proteins, and peptide analogs and derivatives; peptidomimetics; antibodies and antigen binding fragments thereof, nucleic acids; nucleic acid analogs and derivatives; an extract made from biological materials such as bacteria, plants, fungi, or animal cells; animal tissues; naturally occurring or synthetic compositions; and any combinations thereof. Preferably, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds and has a molecular weight of less than 5000 Daltons (5 kDa), preferably less than 3 kDa, still more preferably less than 2 kDa, and most preferably less than 1 kDa. In some cases, it is preferred that a small molecule have a molecular weight equal to or less than 700 Daltons.

As used herein a “peptide” is an oligopeptide, for example, a sequence of 2 to 25 amino acids. The term “peptide”, unless otherwise specified, includes in its scope a peptide that contains an already known analog of a naturally-occurring amino acid having a function as well as the naturally-occurring amino acid. A “protein” comprises one or more peptide (polypeptide) chains and can comprise more amino acids than a peptide. The terms “peptide,” “polypeptide,” and “protein,” may be used interchangeably herein.

Exemplary therapeutic agents include, but are not limited to, those approved by the FDA, subject to a new drug application with the FDA, in clinical trials or in preclinical research.

APIs include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-i-antagonist, an anticholinergic/antispasmodic agent, a vasopressin analogue, an antiarrhythmic agent, an anti-parkinsonian agent, an anti-angina/antihypertensive agent, an anticoagulant agent, an antiplatelet agent, a sedative, an anxiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; anti-inflammatory agents, including anti-asthmatic anti-inflammatory agents, anti-arthritis anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxen, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-i-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; anti-angina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Examples of suitable APIs include infliximab, etanercept, bevacizumab, ranibizumab, adalimumab, certolizumab pegol, golimumab, Interleukin 1 (IL-1) blockers such as anakinra, T cell costimulation blockers such as abatacept, Interleukin 6 (IL-6) blockers such as tocilizumab; Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/.beta.2 blockers such as Anti-lymphotoxin alpha (LTa) or anti-VEGF agents and the like.

Drugs or API include proteins or peptides, including but not limited, monoclonal antibodies (e.g., humanized, human, and/or mouse/human chimeric), polyclonal antibodies, and antibody-drug conjugates. Exemplary peptide/protein therapeutics include insulin, etanercept, pegfilgrastim, salmon calcitonin, cyclosporine, octreotide, liraglutide, bivalirudin, desmopressin, C1 esterase inhibitor (RUCONSET®), human glucocerebrosidase (ELELYSO®), humanized anti-CD20 monoclonal antibody (GAVYZA®), VEGFR Fc-fusion (EYLEA®), glucagon-like peptide-1 receptor agonist Fc-fusion (TRULICITY®), VEGFR Fc-fusion (ZALTRAP), Recombinant factor IX Fc fusion (ALPROLIX), Recombinant factor VIII Fc-fusion (ELOCTATE), GLP-1 receptor agonist-albumin fusion (TANZEUM®), Recombinant factor IX albumin fusion (IDELIVION®), PEGylated IFNb-1a (PLEGRIDY®), Recombinant factor VIII PEGylated (ADYNOVATE®), humanized anti-HER2/neu conjugated to emtansine (KADCYLA®), belimumab, ipilimumab, belatacept, brentuximab vedotin, aflibercept, asparaginase erwinia chrsanthemi, glucarpidase, taliglucerase alfa, pertuzumab, ziv-afilbercept, tbo-filgrastm, ocriplasmin, raxibacumab, ado-trastuzmab emtansine, golimumab, tocilizumab, Obinutuzumab, elosulfase alfa, metreleptin, albiglutide, ramucirumab, siltuxiumab, vedolizumab, peginterferon beta-1a, pembrolizumab, dulaglutide, bintumomab, nivolumab, secukinumab, parathyroid hormone, filgrastim-sndz, dinutuximab, alirocumab, evolocumab, idaracizumab, asfotase-alfa, mepolizumab, dratumumab, necitumumab, elotuzumab, sebelipase alfa, obiltoxaximab, ixekizumab, reslizumab, infliximab-dyyb, atezolizumab, daclizumab, etancerpt-szzs, coagulation factor IX recombinant human, antihemophilic factor (recombinant), coagulation factor XIII A-subunit (recombinant), coagulation factor IX (recombinant), Fc fusion protein, antihemophilic factor (recombinant), Fc fusion protein, C1 esterase inhibitor recombinant, antihemophilic factor porcine, B-domain truncated recombinant, coagulation factor IX (recombinant), antihemophilic factor (recombinant), antihemophilic factor (recombinant) PEGylated, von Willebrand factor (recombinant), coagulation factor IX recombinant human, and antihemophilic factor (recombinant).

The present invention is particularly applicable to the administration of anti-cancer agents. For example, the agent can be a DNA demethylating agents 5-azacytidine (azacitidine) or 5-aza-2′-deoxycytidine (decitabine), (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; Zebularine; 2′,3′-dideoxy-5-fluoro-3′-thiacytidine (Emtriva); 2′-cyclocytidine (Ancitabine); Fazarabine or ara-AC; 6-azacytidine (6-aza-CR); 5,6-dihydro-5-azacytidine (dH-aza-CR); N.sup.4-pentyloxy-carbonyl-5′-deoxy-5-fluorocytidine (Capecitabine); N⁴-octadecyl-cytarabine; or elaidic acid cytarabine. The cytidine analog can also be structurally related to cytidine or deoxycytidine and functionally mimics and/or antagonizes the action of cytidine or deoxycytidine. The agents can also include 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839 or a pharmaceutically acceptable salt thereof.

The anticancer agents include, but are not limited to, an inhibitor, agonist, antagonist, ligand, modulator, stimulator, blocker, activator or suppressor of a gene, ligand, receptor, protein, factor such as an adenosine receptor (such as A2B, A2a, A3), Abelson murine leukemia viral oncogene homolog 1 gene (ABL, such as ABL1), Acetyl-CoA carboxylase (such as ACC1/2), adrenocorticotropic hormone receptor (ACTH), activated CDC kinase (ACK, such as ACK1), Adenosine deaminase, Adenylate cyclase, ADP ribosyl cyclase-1, Aerolysin, Angiotensinogen (AGT) gene, murine thymoma viral oncogene homolog 1 (AKT) protein kinase (such as AKT1, AKT2, AKT3), AKT1 gene, Alkaline phosphatase, Alpha 1 adrenoceptor, Alpha 2 adrenoceptor, Alpha-ketoglutarate dehydrogenase (KGDH), Aminopeptidase N, Arginine deiminase, Beta adrenoceptor, Anaplastic lymphoma kinase receptor, anaplastic lymphoma kinase (ALK, such as ALK1), Alk-5 protein kinase, AMP activated protein kinase, Androgen receptor, Angiopoietin (such as ligand-1, ligand-2), apolipoprotein A-I (APOA1) gene, apoptosis signal-regulating kinase (ASK, such as ASK1), Apoptosis inducing factor, apoptosis protein (such as 1, 2), Arginase (I), asparaginase, Asteroid homolog 1 (ASTEl) gene, ataxia telangiectasia and Rad 3 related (ATR) serine/threonine protein kinase, Axl tyrosine kinase receptor, Aromatase, Aurora protein kinase (such as 1, 2), Basigin, BCR (breakpoint cluster region) protein and gene, B-cell lymphoma 2 (BCL2) gene, Bc12 protein, Bc12 binding component 3, BCL2L11 gene, Baculoviral IAP repeat containing 5 (BIRCS) gene, B-Raf proto-oncogene (BRAF), Brc-Abl tyrosine kinase, Beta-catenin, B-lymphocyte antigen CD19, B-lymphocyte antigen CD20, B-lymphocyte stimulator ligand, B-lymphocyte cell adhesion molecule, Bone morphogenetic protein-10 ligand, Bone morphogenetic protein-9 ligand modulator, Brachyury protein, Bradykinin receptor, Bruton's tyrosine kinase (BTK), Bromodomain and external domain (BET) bromodomain containing protein (such as BRD2, BRD3, BRD4), Calmodulin, calmodulin-dependent protein kinase (CaMK, such as CAMKII), Cancer testis antigen 2, Cancer testis antigen NY-ESO-1, Cannabinoid receptor (such as CB1, CB2), Carbonic anhydrase, caspase 8 apoptosis-related cysteine peptidase CASP8-FADD-like regulator, Caspase (such as caspase-3, caspase-7, Caspase-9), Caspase recruitment domain protein-15, Cathepsin G, chemokine (C-C motif) receptor (such as CCR2, CCR4, CCR5), CCR5 gene, Chemokine CC21 ligand, cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e, CD70 gene, CD74, CD79, CD79b, CD79B gene, CD80, CD95, CD99, CD117, CD122, CDwl23, CD134, CDwl37, CD158a, CD158b1, CD158b2, CD223, CD276 antigen; Chorionic gonadotropin, Cyclin G1, Cyclin D1, cyclin-dependent kinases (CDK, such as CDK1, CDK1B, CDK2-9), casein kinase (CK, such as CM, CMI), c-Kit (tyrosine-protein kinase Kit or CD117), c-Met (hepatocyte growth factor receptor (HGFR)), CDK-activating kinase (CAK), Checkpoint kinase (such as CHK1, CHK2), Cholecystokinin CCK2 receptor, Claudin (such as 6, 18), Clusterin, Complement C3, COP9 signalosome subunit 5, CSF-1 (colony-stimulating factor 1 receptor), CSF2 gene, clusterin (CLU) gene, Connective tissue growth factor, cyclooxygenase (such as 1, 2), cancer/testis antigen 1B (CTAG1) gene, CTLA-4 (cytotoxic T-lymphocyte protein 4) receptor, CYP2B1 gene, Cysteine palmitoyltransferase porcupine, cytokine signalling-1, cytokine signalling-3, Cytochrome P450 11B2, Cytochrome P450 reductase, cytochrome P450 3A4, cytochrome P450 17A1, Cytochrome P450 17, Cytochrome P450 2D6, (provided they anticancer or cytrochrome modifying agents are something other than cobicistat), Cytoplasmic isocitrate dehydrogenase, Cytosine deaminase, cytosine DNA methyltransferase, cytotoxic T-lymphocyte protein-4, chemokine (C-X-C motif) receptor (such as CXCR4, CXCR1 and CXCR2), Delta-like protein ligand (such as 3, 4), Deoxyribonuclease, Dickkopf-1 ligand, Dihydropyrimidine dehydrogenase, DNA binding protein (such as HU-beta), DNA dependent protein kinase, DNA gyrase, DNA methyltransferase, DNA polymerase (such as alpha), DNA primase, discoidin domain receptor (DDR, such as DDR1), DDR2 gene, dihydrofolate reductase (DHFR), Dipeptidyl peptidase IV, L-dopachrome tautomerase, dUTP pyrophosphatase, echinoderm microtubule like protein 4, epidermal growth factor receptor (EGFR) gene, EGFR tyrosine kinase receptor, Eukaryotic translation initiation factor 5A (EIFSA) gene, Elastase, Elongation factor 1 alpha 2, Elongation factor 2, Endoglin, Endonuclease, Endoplasmin, Endosialin, Endostatin, endothelin (such as ET-A, ET-B), Enhancer of zeste homolog 2 (EZH2), epidermal growth factor, epidermal growth factor receptors (EGFR), Epithelial cell adhesion molecule (EpCAM), Ephrin (EPH) tyrosine kinase (such as Epha3, Ephb4), Ephrin B2 ligand, Epigen, Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, Extracellular signal-regulated kinases (ERK), E-selectin, Estradiol 17 beta dehydrogenase, Estrogen receptor (such as alpha, beta), Estrogen related receptor, Exportin 1, Extracellular signal related kinase (such as 1, 2), Factor (such as Xa, VIIa), Fas ligand, Fatty acid synthase, Ferritin, focal adhesion kinase (FAK, such as FAK2), fibroblast growth factor (FGF, such as FGF1, FGF2, FGF4), FGF-2 ligand, FGF-5 ligand, Fibronectin, Fms-related tyrosine kinase 3 (Flt3), farnesoid x receptor (FXR), Folate, Folate transporter 1, Folate receptor (such as alpha), folate hydrolase prostate-specific membrane antigen 1 (FOLH1), paired basic amino acid cleaving enzyme (FURIN), FYN tyrosine kinase, Galactosyltransferase, Galectin-3, glucocorticoid-induced TNFR-related protein GITR receptor, Glucocorticoid, Beta-glucuronidase, Glutamate carboxypeptidase II, glutaminase, Glutathione S-transferase P, Glypican 3 (GPC3), glycogen synthase kinase (GSK, such as 3-beta), Granulocyte-colony stimulating factor (GCSF) ligand, Granulocyte macrophage colony stimulating factor (GM-CSF) receptor, gonadotropin-releasing hormone (GNRH), growth factor receptor-bound protein 2 (GRB2), molecular chaperone groEL2 gene, Grp78 (78 kDa glucose-regulated protein) calcium binding protein, Imprinted Maternally Expressed Transcript (H19) gene, Heat stable enterotoxin receptor, Heparanase, Hepatocyte growth factor, Heat shock protein gene, Heat shock protein (such as 27, 70, 90 alpha, beta), Hedgehog protein, HERV-H LTR associating protein 2, Hexose kinase, tyrosine-protein kinase HCK, Histamine H2 receptor, histone deacetylase (HDAC, such as 1, 2, 3, 6, 10, 11), Histone H1, Histone H3, Histone methyltransferase (DOTIL), Human leukocyte antigen (HLA), HLA class I antigen (A-2 alpha), HLA class II antigen, Homeobox protein NANOG, mitogen-activated protein kinase kinase 1 (MAP4K1, HPK1), HSPB1 gene, Human papillomavirus (such as E6, E7) protein, Hyaluronidase, Hyaluronic acid, Hypoxia inducible factor-1 alpha, Intercellular adhesion molecule 1 (ICAM-1), immunoglobulin (such as G, G1, G2, K, M), indoleamine 2,3-dioxygenase (IDO, such as IDO1), indoleamine pyrrole 2,3-dioxygenase 1 inhibitor, I-Kappa-B kinase (IKK, such as IKK.beta.epsilon.), Immunoglobulin Fc receptor, Immunoglobulin gamma Fc receptor (such as I, III, IIIA), Interleukin 1 ligand, interleukin 2 ligand, Interleukin-2, IL-2 gene, IL-1 alpha, IL-1 beta, IL-2, IL-2 receptor alpha subunit, IL-3 receptor, IL-4, IL-6, IL-7, IL-8, IL-12, IL-15, IL-12 gene, IL-17, Interleukin 13 receptor alpha 2, Interleukin-29 ligand, interleukin-1 receptor-associated kinase 4 (IRAK4), Insulin-like growth factor (such as 1, 2), insulin receptor, Integrin alpha-V/beta-3, Integrin alpha-V/beta-5, Integrin alpha-V/beta-6, Integrin alpha-5/beta-1, Integrin alpha-4/beta-1, integrin alpha-4/beta-7, Interferon inducible protein absent in melanoma 2 (AIM2), interferon (such as alpha, alpha 2, beta, gamma), interferon type I receptor, isocitrate dehydrogenase (such as IDH1, IDH2), Janus kinase (JAK, such as JAK1, JAK2), Jun N terminal kinase, Kinase insert domain receptor (KDR), Killer cell Ig like receptor, Kisspeptin (KISS-1) receptor, v-kit Hardy-Zuckerman 4 feline sarcoma viral oncogene homolog (KIT) tyrosine kinase, KIT gene, Kinesin-like protein KIF 11, kallikrein-related peptidase 3 (KLK3) gene, Kirsten rat sarcoma viral oncogene homolog (KRAS) gene, lactoferrin, lymphocyte activation gene 3 protein (LAG-3), lysosomal-associated membrane protein family (LAMP) gene, Lanosterol-14 demethylase, LDL receptor related protein-1, Leukotriene A4 hydrolase, Listeriolysin, L-Selectin, Luteinizing hormone receptor, Lyase, Lymphocyte antigen 75, lysine demethylases (such as KDM1, KDM2, KDM4, KDM5, KDM6, A/B/C/D), Lymphocyte function antigen-3 receptor, lymphocyte-specific protein tyrosine kinase (LCK), Lymphotactin, Lyn (Lck/Yes novel) tyrosine kinase, Lysophosphatidate-1 receptor, lysyl oxidase protein (LOX), lysyl oxidase-like protein (LOXL, such as LOXL2), Lysyl oxidase homolog 2, Macrophage migration inhibitory fact, melanoma antigen family A3 (MAGEA3) gene, MAGEC1 gene, MAGEC2 gene, Major vault protein, myristoylated alanine-rich protein kinase C substrate (MARCKS) protein, Melan-A (MART-1) melanoma antigen, Mas-related G-protein coupled receptor, matrix metalloprotease (MMP, such as MMP2, MMP9), myeloid cell leukemia 1 (MCL1) gene, Mcl-1 differentiation protein, macrophage colony-stimulating factor (MCSF) ligand, Melanoma associated antigen (such as 1, 2, 3, 6), melanocyte stimulating hormone ligand, Melanocyte protein Pmel 17, Membrane copper amine oxidase, Mesothelin, Metabotropic glutamate receptor 1, mitogen-activated protein kinase (MEK, such as MEK1, MEK2), Hepatocyte growth factor receptor (MET) gene, MET tyrosine kinase, methionine aminopeptidase-2, mitogen-activate protein kinase (MAPK), Mdm2 p53-binding protein, Mdm4 protein, Metalloreductase STEAPI (six transmembrane epithelial antigen of the prostate 1), Metastin, Methyltransferase, Mitochondrial 3 ketoacyl CoA thiolase, MAPK-activated protein kinase (such as MK2), mTOR (mechanistic target of rapamycin (serine/threonine kinase), mTOR complex (such as 1, 2), mucin (such as 1, 5A, 16), mut T homolog (MTH, such as MTH1), Myc proto-oncogene protein, NAD ADP ribosyltransferase, natriuretic peptide receptor C, Neural cell adhesion molecule 1, Neurokinin receptor, Neuropilin 2, Nitric oxide synthase, Nuclear Factor (NF) kappa B, NF kappa B activating protein, Neurokinin 1 (NK1) receptor, NK cell receptor, NK3 receptor, NKG2 A B activating NK receptor, NIMA-related kinase 9 (NEK9), Noradrenaline transporter, Notch (such as Notch-2 receptor, Notch-3 receptor), nucleophosmin-anaplastic lymphoma kinase (NPM-ALK), 2,5-oligoadenylate synthetase, Nuclear erythroid 2-related factor 2, Nucleolin, Nucleophosmin, O-methylguanine DNA methyltransferase, Ornithine decarboxylase, Orotate phosphoribosyltransferase, orphan nuclear hormone receptor NR4A1, Opioid receptor (such as delta), Osteocalcin, Osteoclast differentiation factor, Osteopontin, OX-40 (tumor necrosis factor receptor superfamily member 4 TNFRSF4, or CD134) receptor, 2 oxoglutarate dehydrogenase, purinergic receptor P2X ligand gated ion channel 7 (P2X7), Parathyroid hormone ligand, p53 tumor suppressor protein, P3 protein, Programmed cell death 1 (PD-1), Proto-oncogene serine/threonine-protein kinase (PIM, such as PIM-1, PIM-2, PIM-3), Poly ADP ribose polymerase (PARP, such as PARP1, 2 and 3), p38 kinase, p38 MAP kinase, platelet-derived growth factor (PDGF, such as alpha, beta), P-Glycoprotein (such as 1), Platelet-derived growth factor (PDGF, such as alpha, beta), PKN3 gene, P-Selectin, phosphatidylinositol 3-kinase (PI3K), phosphoinositide-3 kinase (PI3K such as alpha, delta, gamma), phosphorylase kinase (PK), placenta growth factor, Pleiotropic drug resistance transporter, Plexin B1, Polo-like kinase 1, peroxisome proliferator-activated receptors (PPAR, such as alpha, delta, gamma), Preferentially expressed antigen in melanoma (PRAME) gene, Probable transcription factor PML, Programmed cell death ligand 1 inhibitor (PD-L1), Progesterone receptor, prostate specific antigen, Prostatic acid phosphatase, Prostanoid receptor (EP4), proteasome, Protein farnesyltransferase, protein kinase (PK, such as A, B, C), Protein E7, protein tyrosine kinase, Protein tyrosine phosphatase beta, polo-like kinase (PLK), PLK1 gene, Prenyl-binding protein (PrPB), protoporphyrinogen oxidase, Prosaposin (PSAP) gene, phosphatase and tensin homolog (PTEN), Purine nucleoside phosphorylase, Pyruvate kinase (PYK), Pyruvate dehydrogenase (PDH), Pyruvate dehydrogenase kinase, Raf protein kinase (such as 1, B), RAF1 gene, Ras GTPase, Ras gene, 5-Alpha-reductase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Ros1 tyrosine kinase, Runt-related transcription factor 3, 5100 calcium binding protein A9, Sarco endoplasmic calcium ATPase, Gamma-secretase, Secreted frizzled related protein-2, Semaphorin-4D, SL cytokine ligand, Serine protease, Signaling lymphocytic activation molecule (SLAM) family member 7, spleen tyrosine kinase (SYK), Src tyrosine kinase, tumor progression locus 2 (TPL2), serine/threonine kinase (STK), signal transduction and transcription (STAT, such as STAT-1, STAT-3, STAT-5), Second mitochondria-derived activator of caspases (SMAC) protein, smoothened (SMO) receptor, Sodium phosphate cotransporter 2B, Sodium iodide cotransporter, Somatostatin receptor (such as 1, 2, 3, 4, 5), Sonic hedgehog protein, Specific protein 1 (Spi) transcription factor, Sphingomyelin synthase, Sphingosine-1-phosphate receptor-1, Sphingosine kinase (such as 1, 2), SRC gene, STAT3 gene, six-transmembrane epithelial antigen of the prostate (STEAP) gene, Steroid sulfatase, stimulator of interferon genes protein, Stimulator of interferon genes (STING) receptor, Stromal cell-derived factor 1 ligand, SUMO (small ubiquitin-like modifier), Superoxide dismutase, Survivin protein, Synapsin 3, Syndecan-1, Synuclein alpha, serine/threonine-protein kinase (TBK, such as TBK1), TATA box-binding protein-associated factor RNA polymerase I subunit B (TAF1B) gene, T-cell surface glycoprotein CD8, T-cell CD3 glycoprotein zeta chain, T-cell differentiation antigen CD6, T cell surface glycoprotein CD28, Tec protein tyrosine kinase, Tek tyrosine kinase receptor, telomerase, Tenascin, Telomerase reverse transcriptase (TERT) gene, Transforming growth factor (TGF, such as beta) kinase, TGF beta 2 ligand, T-cell immunoglobulin and mucin-domain containing-3 (TIM-3), Tissue factor, Tumor necrosis factor (TNF, such as alpha, beta), TNF related apoptosis inducing ligand, TNFR1 associated death domain protein, TNFSF9 gene, TNFSF11 gene, trophoblast glycoprotein (TPBG) gene, Transferrin, Tropomyosin receptor kinase (Trk) receptor (such as TrkA, TrkB, TrkC), Trophoblast glycoprotein, Thymidylate synthase, Tyrosine kinase with immunoglobulin-like and EGF-like domains (TIE) receptor, Toll-like receptor (TLR such as 1-13), topoisomerase (such as I, II, III), Tumor protein 53 (TP53) gene, Transcription factor, Transferase, Transforming growth factor TGF-.beta. receptor kinase, Transglutaminase, Translocation associated protein, Transmembrane glycoprotein NMB, Tumor necrosis factor 13C receptor, Thymidine kinase, Thymidine phosphorylase, Thymidylate synthase, Thymosin (such as alpha 1), Thyroid hormone receptor, Trop-2 calcium signal transducer, Thyroid stimulating hormone receptor, Tryptophan 5-hydroxylase, Tyrosinase, tyrosine kinase (TK), Tyrosine kinase receptor, Tyrosine protein kinase ABL1 inhibitor, tank-binding kinase (TBK), Thrombopoietin receptor, TNF-related apoptosis-inducing ligand (TRAIL) receptor, Tubulin, Tumor suppressor candidate 2 (TUSC2) gene, Tyrosine hydroxylase, Ubiquitin-conjugating enzyme E21 (UBE2I, UBC9), Ubiquitin, Ubiquitin carboxyl hydrolase isozyme L5, Ubiquitin thioesterase-14, Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase Yes, Wee-1 protein kinase, Wilms' tumor protein, Wilms' tumor antigen 1, X-linked inhibitor of apoptosis protein, Zinc finger protein transcription factor or any combination thereof.

The anticancer agent includes agents defined by their mechanism of action or class, including: anti-metabolites/anti-cancer agents such as pyrimidine analogs floxuridine, capecitabine, cytarabine, CPX-351 (liposomal cytarabine, daunorubicin), TAS-118; purine analogs, folate antagonists (such as pralatrexate), and related inhibitors; antiproliferative/antimitotic agents including natural products such as vinca alkaloid (vinblastine, vincristine) and microtubule such as taxane (paclitaxel, docetaxel), vinblastin, nocodazole, epothilones, vinorelbine) (NAVELBINE), and epipodophyllotoxins (etoposide, teniposide); DNA damaging agents such as actinomycin, amsacrine, busulfan, carboplatin, chlorambucil, cisplatin, cyclophosphamide) (CYTOXAN), dactinomycin, daunorubicin, doxorubicin, epirubicin, iphosphamide, melphalan, merchlorethamine, mitomycin C, mitoxantrone, nitrosourea, procarbazine, taxol, Taxotere, teniposide, etoposide, and triethylenethiophosphoramide; DNA-hypomethylating agent such as guadecitabine (SGI-110) antibiotics such as dactinomycin, daunorubicin, doxorubicin, idarubicin, anthracyclines, mitoxantrone, bleomycins, plicamycin (mithramycin), and; enzymes such as L-asparaginase which systemically metabolizes L-asparagine and deprives cells which do not have the capacity to synthesize their own asparagine; antiplatelet agents; a DNAi oligonucleotide targeting Bcl-2 such as PNT2258; agents that activate or reactivate latent human immunodeficiency virus (HIV) such as panobinostat or romidepsin asparaginase stimulators, such as crisantaspase (ERWINASE®) and GRASPA (ERY-001, ERY-ASP); pan-Trk, ROS1 and ALK inhibitors such as entrectinib anaplastic lymphoma kinase (ALK) inhibitors such as alectinib antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine); antiproliferative/antimitotic antimetabolites such as folic acid analogs (methotrexate); platinum coordination complexes (cisplatin, oxiloplatinim, and carboplatin), procarbazine, hydroxyurea, mitotane, and aminoglutethimide; hormones, hormone analogs (estrogen, tamoxifen, goserelin, bicalutamide, and nilutamide), and aromatase inhibitors (letrozole and anastrozole); anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents (breveldin); immunosuppressives tacrolimus, sirolimus, azathioprine, and mycophenolate; compounds (TNP-470, genistein) and growth factor inhibitors (vascular endothelial growth factor inhibitors, and fibroblast growth factor inhibitors such as FPA14; angiotensin receptor blockers, nitric oxide donors; antisense oligonucleotides, such as AEG35156; DNA interference oligonucleotides, such as PNT2258, AZD-9150 antibodies such as trastuzumab and rituximab; anti-HER3 antibodies, such as LJM716 anti-HER2 antibodies such as margetuximab; anti-HLA-DR antibodies such as INMU-114; anti-IL-3 antibodies, such as JNJ-56022473; anti-OX40 antibodies such as MEDI6469 anti-EphA3 antibodies, such as KB-004; an anti-CD20 antibody such as obinutuzumab; an anti-programmed cell death protein 1 (anti-PD-1) antibody such as nivolumab (OPDIVO, BMS-936558, MDX-1106), pembrolizumab (KEYTRUDA, MK-3477, SCH-900475, lambrolizumab, CAS Reg. No. 1374853-91-4), pidilizumab, and anti-programmed death-ligand 1 (anti-PD-L1) antibodies such as BMS-936559, atezolizumab (MPDL3280A), durvalumab (MEDI4736), avelumab (MSB0010718C), and MDX1105-01, CXCR4 antagonists such as BL-8040; CXCR2 antagonist such as AZD-5069; GM-CSF antibodies such as lenzilumab. Selective estrogen receptor downregulator (SERD) such as fulvestrant (Faslodex); a transforming growth factor-beta (TGF-beta) kinase antagonist such as galunisertib; a bispecific antibody such as MM-141 (IGF-1/ErbB3), MM-111 (Erb2/Erb3), JNJ-64052781 (CD19/CD3). Mutant selective EGFR inhibitors, such as PF-06747775, EGF816, ASP8273, ACEA-0010, BI-1482694. Alpha-ketoglutarate dehydrogenase (KGDH) inhibitors such as CPI-613, XPO1 inhibitors such as selinexor (KPT-330). Isocitrate dehydrogenase 2 (IDH2) inhibitors such as enasidenib (AG-221), and IDH1 inhibitors such as AG-120, and AG-881 (IDH1 and IDH2). Agents that target the interleukin-3 receptor (IL-3R) such as SL-401. Arginine deiminase stimulators, such as pegargiminase (ADI-PEG-20) antibody-drug conjugates, such as MLN0264 (anti-GCC, guanylyl cyclase C), T-DM1 (trastuzumab emtansine, Kadcycla), milatuzumab-doxorubicin (hCD74-DOX), brentuximab vedotin, DCDT2980S, polatuzumab vedotin, SGN-CD70A, SGN-CD19A, inotuzumab ozogamicin, lorvotuzumab mertansine, SAR3419, isactuzumab govitecan, anti-claudin-18.2 antibodies such as IMAB362.beta.-catenin inhibitors, such as CWP-291 a CD73 antagonist such as MEDI-9447; c-PIM inhibitors, such as PIM447, a BRAF inhibitor such as dabrafenib, vemurafenib, a sphingosine kinase-2 (SK2) inhibitor such as Yeliva. (ABC294640) cell cycle inhibitors such as selumetinib (MEK1/2), sapacitabine, AKT inhibitors such as MK-2206, ipatasertib, afuresertib, anti-CTLA-4 (cytotoxic T-lymphocyte protein-4) inhibitor such as tremelimumab, c-MET inhibitors, such as AMG-337, savolitinib, tivantinib (ARQ-197), capmatinib, tepotinib inhibitors of CSFIR/KIT and FLT3 such as PLX3397, a kinase inhibitor such as vandetanib; E selectin antagonists such as GMI-1271, differentiation inducers such as tretinoin; epidermal growth factor receptor (EGFR) inhibitors such as osimertinib (AZD-9291) topoisomerase inhibitors (doxorubicin, daunorubicin, dactinomycin, eniposide, epirubicin, etoposide, idarubicin, irinotecan, mitoxantrone, pixantrone, sobuzoxane, topotecan, and irinotecan, MM-398 (liposomal irinotecan), vosaroxin and corticosteroids (cortisone, dexamethasone, hydrocortisone, methylprednisolone, prednisone, and prednisolone); growth factor signal transduction kinase inhibitors; dysfunction inducers; nucleoside analogs such as DFP-10917 Axl inhibitors such as BGB-324; BET inhibitors such as INCB-054329, PARP inhibitors such as olaparib, rucaparib, veliparib, Proteasome inhibitors such as ixazomib, carfilzomib (Kyprolis); Glutaminase inhibitors such as CB-839; vaccines such as peptide vaccine TG-01 (RAS), bacterial vector vaccines such as CRS-207/GVAX, autologous Gp96 vaccine, dendritic cells vaccines, Oncoquest-L vaccine, DPX-Survivac, ProstAtak, DCVAC, ADXS31-142, demcizumab (anti-DLL4, Delta-like ligand 4, Notch pathway), napabucasin (BBI-608) smoothened (SMO) receptor inhibitors, such as ODOMZO® (sonidegib, formerly LDE-225), LEQ506, vismodegib (GDC-0449), BMS-833923, glasdegib (PF-04449913), LY2940680, and itraconazole; interferon alpha ligand modulators, such as interferon alfa-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus) (Inmutag, Inter 2A), interferon alfa-2b follow-on biologic (Biosidus-Bioferon, Citopheron, Ganapar) (Beijing Kawin Technology-Kaferon) (AXXO-interferon alfa-2b), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-nl (Humoferon, SM-10500, Sumiferon); interferon gamma ligand modulators, such as interferon gamma (OH-6000, Ogamma 100); IL-6 receptor modulators, such as tocilizumab, siltuximab, AS-101 (CB-06-02, IVX-Q-101); Telomerase modulators, such as tertomotide (GV-1001, HR-2802, Riavax) and imetelstat (GRN-163, JNJ-63935937) DNA methyltransferases inhibitors, such as temozolomide (CCRG-81045), decitabine, guadecitabine (S-110, SGI-110), KRX-0402, and azacitidine; DNA gyrase inhibitors, such as pixantrone and sobuzoxane; Bcl-2 family protein inhibitor ABT-263, venetoclax (ABT-199), ABT-737, and AT-101; Notch inhibitors such as LY3039478, tarextumab (anti-Notch2/3), BMS-906024 anti-myostatin inhibitors such as landogrozumab, hyaluronidase stimulators such as PEGPH-20, Wnt pathway inhibitors such as SM-04755, PRI-724, gamma-secretase inhibitors such as PF-03084014, IDO inhibitors such as indoximod, Grb-2 (growth factor receptor bound protein-2) inhibitor BP1001 (liposomal Grb-2), TRAIL pathway-inducing compounds, such as ONC201, Focal adhesion kinase inhibitors such as VS-4718, defactinib, hedgehog inhibitors such as saridegib, sonidegib (LDE225), glasdegib and vismodegib, Aurora kinase inhibitors such as alisertib (MLN-8237), modulators of HSPB1 activity (heat shock protein 27, HSP27), such as brivudine, apatorsen, ATR inhibitor such as AZD6738, and VX-970, mTOR inhibitors, such as sapanisertib, Hsp90 inhibitors such as AUY922. Murine double minute (mdm2) oncogene inhibitors such as DS-3032b CD137 agonist such as urelumab, Anti-KIR monoclonal antibodies such as lirilumab (IPH-2102). Antigen CD19 inhibitors such as MOR208, MEDI-551, AFM-11, CD44 binders such as A6, CYP17 inhibitors, such as VT-464, ASN-001, ODM-204. RXR agonists such as IRX4204, TLRs (Toll-like receptors) agonists such as IMO-8400 A hedgehog/smoothened (hh/Smo) antagonist such as taladegib. Immunomodulators such as complement C3 modulators, such as Imprime PGG. Intratumural immune-oncology agents such as G100 (TLR4 agonist) IL-15 agonists such as ALT-803 EZH2 (enhancer of zeste homolog 2) inhibitors such as tazemetostat. Oncolytic viruses, such as pelareorep, and talimogene laherparepvec). DOT1L (histone methyltransferase) inhibitors such as pinometostat (EPZ-5676), toxins such as Cholera toxin, ricin, Pseudomonas exotoxin, Bordetella pertussis adenylate cyclase toxin, diphtheria toxin, and caspase activators; and chromatin. DNA plasmid such as BC-819. PLK inhibitors of PLK 1, 2, and 3, such as volasertib (PLK1). Apoptosis Signal-Regulating Kinase (ASK) Inhibitors: ASK inhibitors include ASK1 inhibitors. Examples of ASK1 inhibitors include, but are not limited to, those described in WO 2011/008709 (Gilead Sciences) and WO 2013/112741 (Gilead Sciences). Bruton's Tyrosine Kinase (BTK) Inhibitors: Examples of BTK inhibitors include, but are not limited to, (S)-6-amino-9-(1-(but-2-ynoyl)pyrrolidin-3-yl)-7-(4-phenoxyphenyl)-7H-pur-in-8(9H)-one, acalabrutinib (ACP-196), BGB-3111, HM71224, ibrutinib, M-2951, ONO-4059, PRN-1008, spebrutinib (CC-292), TAK-020. Cyclin-dependent Kinase (CDK) Inhibitors: CDK inhibitors include inhibitors of CDK 1, 2, 3, 4, 6 and 9, such as abemaciclib, alvocidib (HMR-1275, flavopiridol), AT-7519, FLX-925, LEE001, palbociclib, ribociclib, rigosertib, selinexor, UCN-01, and TG-02. Discoidin Domain Receptor (DDR) Inhibitors: DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations). Histone Deacetylase (HDAC) Inhibitors: Examples of HDAC inhibitors include, but are not limited to, abexinostat, ACY-241, AR-42, BEBT-908, belinostat, CKD-581, CS-055 (HBI-8000), CUDC-907, entinostat, givinostat, mocetinostat, panobinostat, pracinostat, quisinostat (JNJ-26481585), resminostat, ricolinostat, SHP-141, valproic acid (VAL-001), vorinostat. Janus Kinase (JAK) Inhibitors: JAK inhibitors inhibit JAK1, JAK2, and/or JAK3. Examples of JAK inhibitors include, but are not limited to, AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB1518), peficitinib (ASP015K), ruxolitinib, tofacitinib (formerly tasocitinib), and XL019. Lysyl Oxidase-Like Protein (LOXL) Inhibitors: LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences). Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics). Matrix Metalloprotease (MMP) Inhibitors: MMP inhibitors include inhibitors of MMP1 through 10. Examples of MMP9 inhibitors include, but are not limited to, marimastat (BB-2516), cipemastat (Ro 32-3555) and those described in WO 2012/027721 (Gilead Biologics). Mitogen-activated Protein Kinase (MEK) Inhibitors: MEK inhibitors include antroquinonol, binimetinib, cobimetinib (GDC-0973, XL-518), MT-144, selumetinib (AZD6244), sorafenib, trametinib (GSK1120212), uprosertib+trametinib. Phosphatidylinositol 3-kinase (PI3K) Inhibitors: PI3K inhibitors include inhibitors of PI3K.gamma., PI3K.delta., PI3.beta., PI3K.alpha., and/or pan-PI3K. Examples of PI3K inhibitors include, but are not limited to, ACP-319, AEZA-129, AMG-319, AS252424, BAY 10824391, BEZ235, buparlisib (BKM120), BYL719 (alpelisib), CH5132799, copanlisib (BAY 80-6946), duvelisib, GDC-0941, GDC-0980, GSK2636771, GSK2269557, idelalisib (ZYDELIG®), IPI-145, IPI-443, KAR4141, LY294002, Ly-3023414, MLN1117, OXY111A, PA799, PX-866, RG7604, rigosertib, RP5090, taselisib, TG100115, TGR-1202, TGX221, WX-037, X-339, X-414, XL147 (SAR245408), XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences). Spleen Tyrosine Kinase (SYK) Inhibitors: Examples of SYK inhibitors include, but are not limited to, 6-(1H-indazol-6-yl)-N-(4-morpholinophenyl)imidazo[1,2-alpyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Conn.). and those described in U.S. 2015/0175616. Tyrosine-kinase Inhibitors (TKIs): TKIs may target epidermal growth factor receptors (EGFRs) and receptors for fibroblast growth factor (FGF), platelet-derived growth factor (PDGF), and vascular endothelial growth factor (VEGF). Examples of TKIs include, but are not limited to, afatinib, bosutinib, brigatinib, cabozantinib, crenolanib, dacomitinib, dasatinib, dovitinib, E-6201, erlotinib, gefitinib, gilteritinib (ASP-2215), HM61713, icotinib, imatinib, KX2-391 (Src), lapatinib, lestaurtinib, midostaurin, nintedanib, osimertinib (AZD-9291), ponatinib, poziotinib, quizartinib, radotinib, rociletinib, sunitinib, and TH-4000. Further anticancer agents include: alkylating agents such as thiotepa and cyclophosphamide (CYTOXAN); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, especially bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlomaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiIl), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel) (TAXOL), abraxane, docetaxel) (TAXOTERE), cabazitaxel, BIND-014; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine) (GEMZAR®); 6-thioguanine; mercaptopurine; methotrexate; vinblastine; platinum; etoposide (VP-16); ifosfamide; mitroxantrone; vancristine; vinorelbine) (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan); and pharmaceutically acceptable salts, acids, or derivatives of any of the above.

Also included in the definition of anticancer agents are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX), raloxifene, droloxifene, 4-hydroxytamoxifen, trioxifene, keoxifene, LY117018, onapristone, and toremifene) (FARESTON). Inhibitors of the enzyme aromatase regulate estrogen production in the adrenal glands. Examples include 4(5)-imidazoles, aminoglutethimide, megestrol acetate) (MEGACE), exemestane, formestane, fadrozole, vorozole) (RIVISOR), letrozole) (FEMARA), and anastrozole) (ARIMIDEX). Examples of anti-androgens include apalutamide, abiraterone, enzalutamide, flutamide, galeterone, nilutamide, bicalutamide, leuprolide, goserelin, ODM-201, APC-100, ODM-204. Examples of progesterone receptor antagonist include onapristone.

Anti-angiogenic agents include, but are not limited to, retinoid acid and derivatives thereof, 2-methoxyestradiol, ANGIOSTATIN, ENDOSTATIN, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, .alpha.,.alpha.′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone. Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethy)dithio)butanoic acid, p-2-amino-3-methyl-3-((2-aminoethy)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

The API can be an immunotherapeutic agent. Immunotherapeutic agents include, and are not limited to, therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, dinutuximab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, pasudotox, narnatumab, naptumomab, necitumumab, nimotuzumab, nofetumomab, obinutuzumab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, ramucirumab (CYRAMZA®) rilotumumab, rituximab, robatumumab, samalizumab, satumomab, sibrotuzumab, siltuximab, solitomab, tacatuzumab, taplitumomab, tenatumomab, teprotumumab, tigatuzumab, tositumomab, trastuzumab, ABP-980, tucotuzumab, ubilituximab, veltuzumab, vorsetuzumab, votumumab, zalutumumab, CC49, OBI-833 and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131.

In addition to the BBB-crossing ligand, the composition can comprise, in place of an API or in addition thereto, a targeting moiety, such as a peptide or protein ligand or domain, covalently or non-covalently attached to the surface of the nanoparticles, which targeting moiety specifically or preferentially binds to a target site within the brain (such as a cell surface receptor on a brain tumor), such that the nanoparticle bearing such a targeting moiety will be specifically or preferentially directed to the target site in vivo. The targeting moiety bearing nanoparticle may further comprise an API that is encapsulated or embedded within the nanoparticle that can be released or otherwise effective at the target site.

By having targeting moieties, target specific nanoparticles are able to efficiently bind to or otherwise associate with a biological entity, for example, a membrane component or cell surface receptor. Targeting of a therapeutic agent (e.g., to a particular tissue or cell type, to a specific diseased tissue but not to normal tissue, etc.) is desirable for the treatment of tissue specific diseases such as cancer (e.g. glioblastoma). For example, in contrast to systemic delivery of a cytotoxic anti-cancer agent, targeted delivery could prevent the agent from killing healthy cells. Additionally, targeted delivery would allow for the administration of a lower dose of the agent, which could reduce the undesirable side effects commonly associated with traditional chemotherapy. As discussed above, the target specificity of the nanoparticles of the invention will be maximized by optimizing the ligand density on the nanoparticle. Targeting moieties can be covalently or non-covalently bound to the surface of the nanoparticle or microparticle. For example, targeting moieties can be covalently bound to the anionic polymer (e.g., by coupling one or more carboxylic acid or other function group moieties), the PLGA/PLA (e.g., via a polymer terminal) or by incorporating yet another molecule or polymer into the interpenetrating network. For example, the targeting moiety can be covalently linked to a polyethylene glycol (PEG) molecule or PLGA-PEG diblock and added to the emulsion with the anionic polymer.

For example, a targeting moiety can bind to or otherwise associate with a biological entity, for example, a cell surface receptor. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

In preferred embodiments, the targeting moiety of the invention is a small molecule. In certain embodiments, the term “small molecule” refers to organic compounds, whether naturally-occurring or artificially created (e.g., via chemical synthesis) that have relatively low molecular weight and that are not proteins, polypeptides, or nucleic acids. Small molecules typically have multiple carbon-carbon bonds. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol.

### Production of the Nanoparticles

The invention described herein provides several basic methods for the preparation of nanoparticles that present BBB-crossing ligands on their surfaces.

The nanoparticles can be manufactured from the coprecipitation or coacervation of a hydrophobic and/or neutral biocompatible polymer, such as PLGA or PBCA, and the BBB-crossing agents. Without being bound by any theory, it is believed that the polymer backbones intertwine or interlace while in the organic phase of emulsion.

As used herein, “small (amount)” refers to a relatively small amount/volume of the first solution of the second solvent as compared to the volume of the first solvent with the biodegradable polymer, such that emulsification of the first solution of the second solvent in the polymer solution in the first solvent forms an emulsion (i.e., the first emulsion) with the continuous phase being the polymer solution. Typically, the volume ratio between the small amount of the first solution of the second solvent, and the first solvent, is at least about 1:n, wherein n can be 1, 2, 3, 4, 5, 6, 7, 8, 9, 10, 20, 30, 40, 50, 60, 70, 80, 90, or 100.

As used herein, “large (amount)” refers to the relatively large amount/volume of the second solution of the second solvent as compared to the volume of the first emulsion, such that emulsification of the first emulsion in the second solution of the second solvent forms an emulsion (i.e., the second emulsion) with the continuous phase being the second solution of the second solvent. Typically, the volume ratio between the first emulsion and the large amount of the second solution of the second solvent, is at least about 1:m, wherein m can be 1, 2, 3, 4, 5, 6, 7, 8, 9, 10, 20, 30, 40, 50, 60, 70, 80, 90, or 100.

Using the methods of preparation described herein, the BBB-crossing agent is tightly integrated into the produced nanoparticles.

The incorporation of the BBB-crossing agent into the nanoparticles can be stable and tight. Thus, preferably, the method further comprises washing said nanoparticles, and/or concentrating said nanoparticles to a desired volume.

An emulsion process may be used to prepare the nanoparticles described herein. The invention includes a method for the preparation of nanoparticles presenting BBB-crossing ligands on their surfaces comprising: (1) dissolving a biodegradable polymer (and optionally an active agent, such as a pharmaceutical ingredient (API), or a poorly water soluble compound) in a first solvent to form a polymer solution; (2) emulsifying the polymer solution in a solution of a second solvent to form an emulsion, wherein the first solvent is not miscible or partially miscible with the second solvent, and wherein the solution of the second solvent comprises a BBB-crossing agent/(ligand), said solution of the second solvent optionally further comprising a surfactant and/or an API soluble in the second solvent; and, (3) removing the first solvent to form said nanoparticles having the surface BBB-crossing ligand.

The invention also provides a double-emulsification method for the preparation of nanoparticles having surface BBB-crossing ligands, said method comprising: (1) dissolving a biodegradable polymer (and optionally an active agent, an API, or a poorly water soluble compound) in a first solvent to form a polymer solution; (2) adding a second solvent to the polymer solution to form a mixture, wherein the first solvent is not miscible or partially miscible with the second solvent, and wherein the first solution of the second solvent optionally comprises an active agent which may be the same or different from the API dissolved in the first solvent; (3) emulsifying the mixture to form a first emulsion; (4) emulsifying the first emulsion in a second solution of the second solvent to form a second emulsion, wherein the second solution of the second solvent comprises a BBB-crossing agent, and optionally further comprises a surfactant; and, (5) removing the first solvent to form nanoparticles having surface BBB-crossing ligand.

Preferably, in the emulsification process, the weight ratio of the polymer solution to the aqueous solution is typically from 1:1,000 to 10:1, preferable from 1:100 to 1:1.

As used herein, miscibility is defined to be the property of liquids to mix in all proportions, forming a homogeneous solution. Substances/liquids are said to be immiscible or not miscible, if in some proportion, they do not form a solution.

Exemplary solvents miscible with water include acetone, tetrahydrofuran (THF), acetonitrile, dimethyl sulfoxide (DMSO), dimethylformamide (DMF).

The double emulsion process may be particularly useful when an active agent, such as a drug or an active pharmaceutical ingredient (API), such as a protein-based therapeutic prepared in an aqueous solution, is first emulsified with a pharmaceutically acceptable polymer solution to form a first emulsion such that the API is encapsulated within the polymer solution. Then the polymer, and the therapeutics encapsulated therein, is again emulsified in a larger volume of solvent to form a second emulsion (e.g., the water-in-oil-in-water or w/o/w type double emulsion), before the nanoparticle is formed.

For example, in the above described w/o/w technique, a relatively small amount of a first solution of the second solvent (e.g., an aqueous protein solution) (e.g., about 20%, 15%, 10%, 5% v/v of the organic solvent) may be introduced into a relatively larger amount of a first solvent (e.g., an organic solvent), such as methylene chloride or ethyl acetate, that dissolves the hydrophobic biodegradable polymer. The first emulsion is then formed using a suitable method, e.g., probe sonication, homogenization or microfluidization. After formation of the first emulsion, a second emulsion is formed by introducing the first emulsion into a larger volume of a second solution of the second solvent (e.g., about at least about 2-fold, 3-fold, 4-fold, 5-fold, 6-fold, 10-fold of the first emulsion) containing an emulsifier, e.g., polyvinyl alcohol. Again, a homogenization method can be used to form the second emulsion. This is next followed by a period of solvent evaporation leading to the hardening of the polymer, typically by stirring for some hours. As a result, the protein solution is trapped into the relative hydrophobic matrix of the biodegradable polymer forming small inclusions. Finally, the nanoparticles formed are collected, washed (e.g., with distilled water) via repeated centrifugation or filtration, followed by dehydration, typically by lyophilization.

In any of the aspects described above, preferably, the first solvent is methylene chloride, ethyl acetate, or chloroform. Preferably, the second solution of the second solvent comprises the BBB-crossing ligand and a surfactant comprising organic or inorganic pharmaceutical excipients; various polymers; oligomers; natural products; nonionic, cationic, zwitterionic, or ionic surfactants; and mixtures thereof. The surfactant may comprise polyvinyl alcohol (PVA), polyvinylpyrrolidone (PVP), a polysorbate (Tween series) surfactant, a PEO—PPO-PEO polyethylene oxide polypropylene oxide triblock copolymer (Pluronic series or Poloxamer series) surfactant, or a t-octylphenyl-polyethylene glycol (Triton X-100) surfactant or a salt, derivative, copolymer, or mixture thereof. Preferably, the surfactant is PVA (see examples).

Preferably, the emulsifying step comprises homogenization, mechanical stirring, and/or microfluidization.

Preferably, the first solvent is removed through solvent exchange and/or evaporation.

The solvent used in the dissolving step for the polymer can be any type of solvent that dissolves the biodegradable polymer (e.g., PLGA). However, a volatile solvent is preferably used for its removal. For example, preferred solvents for forming the PLGA solution include methylene chloride, ethyl acetate, and chloroform.

In the emulsifying step, the (aqueous) solution may contain a surfactant or surface stabilizer. Surfactants generally include compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, which contain both hydrophobic groups (usually branched, linear, or aromatic hydrocarbon chain(s), fluorocarbon chain(s), or siloxane chain(s) as “tail(s)”) and hydrophilic groups (usually heads). Surfactants are most commonly classified according to their polar head group: a non-ionic surfactant has no charge groups in its head; an ionic surfactant carries a net charge —if the charge is negative, the surfactant is anionic, and if the charge is positive, it is cationic. If a surfactant contains a head with two oppositely charged groups, it is termed zwitterionic. Preferably, anionic or zwitterionic surfactants, such as those containing carboxyl groups (“carboxylates”), are preferably used in the instant invention. The carboxylates are the most common surfactants and comprise the alkyl carboxylates, such as sodium stearate, sodium lauroyl sarcosinate, and carboxylate-based fluorosurfactants such as perfluorononanoate, perfluorooctanoate (PFOA or PFO).

While not wishing to be bound by any particular theory, surfactant may be useful for the formation and stabilization of the emulsion droplets. The surfactant may also comprise organic or inorganic pharmaceutical excipients, various polymers, oligomers, natural products, nonionic, cationic, zwitterionic, or ionic surfactants, and mixtures thereof.

The surfactants that can be used for the preparation of the subject nanoparticles include polyvinyl alcohol, polyvinylpyrrolidone, Tween or polysorbate series, Pluronic series, Poloxamer series, Triton X-100, etc. Additional suitable surfactants are provided herein below.

The emulsification process may be carried out by any art-recognized means, such as homogenization, ultrasonication, mechanical stirring, microfluidization, or a combination thereof.

The removal of solvent is usually achieved by, for example, solvent exchange and evaporation.

Combinations of more than one surfactant can be used in the invention. Useful surfactants or surface stabilizers which can be employed in the invention may include, but are not limited to, known organic and inorganic pharmaceutical excipients. Such excipients include various polymers, low molecular weight oligomers, natural products, and surfactants. Surfactants or surface stabilizers include nonionic, cationic, zwitterionic, and ionic surfactants.

Representative examples of other useful surfactants or surface stabilizers include hydroxypropyl methylcellulose, hydroxypropylcellulose, polyvinylpyrrolidone, sodium lauryl sulfate, sodium dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available TWEENS® such as e.g., TWEEN 20® and TWEEN 80® (ICI Specialty Chemicals)); polyethylene glycols (e.g., CARBOWAXS 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., PLURONICS F68® and F108®, which are block copolymers of ethylene oxide and propylene oxide); poloxamines (e.g., TETRONIC 908®, also known as POLOXAMINE 908®, which is a tetrafunctional block copolymer derived from sequential addition of propylene oxide and ethylene oxide to ethylenediamine (BASF Wyandotte Corporation, Parsippany, N.J.)); TETRONIC 1508® (T-1508) (BASF Wyandotte Corporation), TRITONS X-200®, which is an alkyl aryl polyether sulfonate (Rohm and Haas); CRODESTAS F-110®, which is a mixture of sucrose stearate and sucrose distearate (Croda Inc.); p-isononylphenoxypoly-(glycidol), also known as OLIN-lOG® or SURFACTANT 10-G® (Olin Chemicals, Stamford, Conn.); Crodestas SL-40(Croda, Inc.); and SA90HCO, which is C18H37CH2(CON(CH3)-CH2(CHOH)4(CH2OH)2 (Eastman Kodak Co.); decanoyl-N-methylglucamide; n-decyl μ-D-glucopyranoside; n-decyl β-D-maltopyranoside; n-dodecyl β-D-glucopyranoside; n-dodecyl β-D-maltoside; heptanoyl-N-methylglucamide; n-heptyl-p-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

Examples of useful cationic surfactants or surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt) (also known as DPPE-PEG(2000)-Amine Na) (Avanti Polar Lipids, Alabaster, A1), Poly(2-methacryloxyethyl trimethylammonium bromide) (Polysciences, Inc., Warrington, Pa.) (also known as S1001), poloxamines such as TETRONIC 908®, also known as POLOXAMINE 908®, which is a tetrafunctional block copolymer derived from sequential addition of propylene oxide and ethylene oxide to ethylenediamine (BASF Wyandotte Corporation, Parsippany, N.J.), lysozyme, long-chain polymers such as alginic acid, carrageenan (FMC Corp.), and POLYOX (Dow, Midland, Mich.).

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C12-15dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy) ammonium chloride or bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12, C15, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surfactants or surface stabilizers and other useful cationic surfactants or surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990), each of which is incorporated by reference herein in its entirety.

Nonpolymeric cationic surfactants or surface stabilizers are any nonpolymeric compound, such as benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quaternary ammonium compounds of the formula NR1R2R3R4(+). For compounds of the formula NR1R2R3R4(+): (i) none of R1-R4 are CH3; (ii) one of R1-R4 is CH3; (iii) three of R1-R4 are CH3; (iv) all of R1-R4 are CH3; (v) two of R1-R4 are CH3, one of R1-R4 is C6H5CH2, and one of R1-R4 is an alkyl chain of seven carbon atoms or less; (vi) two of R1-R4 are CH3, one of R1-R4 is C6H5CH2, and one of R1-R4 is an alkyl chain of nineteen carbon atoms or more; (vii) two of R1-R4 are CH3 and one of R1-R4 is the group C6H5 (CH2)n, where n>1; (viii) two of R1-R4 are CH3, one of R1-R4 is C6H5CH2, and one of R1-R4 comprises at least one heteroatom; (ix) two of R1-R4 are CH3, one of R1-R4 is C6H5CH2, and one of R1-R4 comprises at least one halogen; (x) two of R1-R4 are CH3, one of R1-R4 is C6H5CH2, and one of R1-R4 comprises at least one cyclic fragment; (xi) two of R1-R4 are CH3 and one of R1-R4 is a phenyl ring; or (xii) two of R1-R4 are CH3 and two of R1-R4 are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

Many surfactants or surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The surfactants or surface stabilizers are commercially available and/or can be prepared by techniques known in the art.

Preferably, the surface of the subject nanoparticle is composed of a material that minimizes nonspecific or unwanted biological interactions between the particle surface and the interstitium, e.g., the particle surface may be coated with a material to prevent or decrease non-specific interactions. Steric stabilization by coating particles with hydrophilic layers such as poly(ethylene glycol) (PEG) and its copolymers such as PLURONICS (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections.

As used herein, “small (amount)” refers to a relatively small amount/volume of the first solution of the second solvent as compared to the volume of the first solvent with the biodegradable polymer (e.g. PLGA or PBCA), such that emulsification of the first solution of the second solvent in the polymer solution in the first solvent forms an emulsion (i.e., the first emulsion) with the continuous phase being the polymer solution. Typically, the volume ratio between the small amount of the first solution of the second solvent, and the first solvent, is at least about 1:n, wherein n can be 1, 2, 3, 4, 5, 6, 7, 8, 9, 10, 20, 30, 40, 50, 60, 70, 80, 90, or 100.

As used herein, “large (amount)” refers to the relatively large amount/volume of the second solution of the second solvent as compared to the volume of the first emulsion, such that emulsification of the first emulsion in the second solution of the second solvent forms an emulsion (i.e., the second emulsion) with the continuous phase being the second solution of the second solvent. Typically, the volume ratio between the first emulsion and the large amount of the second solution of the second solvent, is at least about 1:m, wherein m can be 1, 2, 3, 4, 5, 6, 7, 8, 9, 10, 20, 30, 40, 50, 60, 70, 80, 90, or 100.

The incorporation of the BBB-crossing ligand into the particles can be stable and tight. Thus, preferably, the method further comprises washing nanoparticles, and/or concentrating said nanoparticles to a desired volume.

The nanoparticles produced using the methods of the invention may routinely undergo washing as part of a purification process that removes impurity, and/or concentrates the nanoparticles so produced.

The nanoparticles produced using the methods of the invention may also undergo more stringent washing tests, e.g., as part of the quality control process, to ensure that the BBB-crossing ligands are stably incorporated into the nanoparticles so produced, and that the amount of free BBB-crossing agents in the nanoparticle suspension is less than 1 mg/ml, less than 0.1 mg/ml, or less than 0.01 mg/ml.

Preferably, the washing test uses conditions identical to or similar to those exemplified below. Preferably, said BBB-crossing agent is durably attached to the surface of the nanoparticles and can sustain multiple washing cycles.

Preferably, the BBB-crossing ligands on particle surface can sustain certain washing tests, such as the wash test exemplified herein, without significantly losing the amount of the BBB-crossing agent.

Preferably, after washing, the nanoparticles retain at least about 50%, 60%, 75%, 80%, 85%, 90%, 95%, or 99% of the amount of the BBB-crossing agent.

### Particle Sizes

The size of the subject nanoparticles is from about 1 nm to about 1000 nm, preferably from about 10 nm to about 500 nm.

As used herein, particle size can be determined by any conventional particle size measuring techniques well known to those skilled in the art. Such techniques include, for example, sedimentation field flow fractionation, photon correlation spectroscopy, light scattering, dynamic light scattering, light diffraction, and disk centrifugation.

### Additional Components

The particles of the present invention may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez et al., Science, 1998, 281:2013; Niemeyer, C. M., Angew. Chem. Int. Ed., 2003, 42:5796; Waggoner, A. Methods Enzymol., 1995, 246:362; Brus, L. E., J. Chem. Phys., 1993, 79, 5566). Quantum dots, such as hybrid organic/inorganic quantum dots based on a class of polymers known as dendrimers, may be used in biological labeling, imaging, and optical biosensing systems (Lemon et al., J. Am. Chem. Soc., 2000, 122:12886). Unlike the traditional synthesis of inorganic quantum dots, the synthesis of these hybrid quantum dot nanoparticles does not require high temperatures or highly toxic, unstable reagents. (Etienne et al., Appl. Phys. Lett., 87:181913, 2005).

### Exemplary Uses

The nanoparticles and compositions thereof that have numerous applications including in therapeutic methods.

Preferably, the nanoparticles or the composition comprising the nanoparticles can be used in a method of treating a disease or condition in a subject in need thereof, or a method of reducing the duration or severity of the disease or condition in the subject in need thereof, wherein the disease or condition is treatable with the nanoparticles (and optionally with a specific API), comprising administering a composition or a pharmaceutical composition comprising the nanoparticles to the subject, thereby treating the disease or condition. Where the nanoparticles comprise (for example, encapsulate) an API, the nanoparticles can be used in a method of administering or delivering the API to a subject in need thereof and/or for a method of treating a subject suffering from a disease or condition that can be treated with the API. For example, when the API is an anti-inflammatory agent, the particles can be administered to a subject from an inflammatory condition.

In additional aspects, the nanoparticles comprise an immunotherapeutic agent and can be used in immunotherapy.

The microparticles and nanoparticles described herein can be used to treat an inflammatory condition. Examples of such diseases and conditions include, but are not limited to, Alzheimer's disease, Parkinson's Disease, meningitis, encephalitis, multiple sclerosis, cerebral infarction, cerebral embolism, Guillame-Barre syndrome, neuritis, neuralgia, spinal cord injury, paralysis, uveitis, cancers, tumors and proliferative disorders (such as brain tumors, including, e.g., glioma, anaplastic oligodendroglioma, adult glioblastoma multiforme, and adult anaplastic astrocytoma).

Any of the methods of treatment provided may be used to treat cancer at various stages. By way of example, the cancer stage includes but is not limited to early, advanced, locally advanced, remission, refractory, reoccurred after remission and progressive.

Preferably, the nanoparticle of the invention can be used in combination with a second therapeutic that is effective for treating any one of the treatable conditions.

Preferably, the subject is a human patient. Preferably, the subject is a non-human mammal, such as a non-human primate, a livestock animal (horse, mule, cattle, bull, cow, sheep, goat, pig, camel, etc.), a rodent (rabbit, hamster, mouse, rat, etc.), or a pet (cat, dog).

Preferably, the method includes administering the subject composition or pharmaceutical composition comprising the subject nanoparticles by any suitable means or routes, such as systemic administrations, preferably intravenously.

Preferably, about 10² to about 10²⁰ particles are provided to the individual. Preferably, between about 10³ to about 10¹⁵ particles are provided. Preferably, between about 10⁶ to about 10¹² particles are provided. Preferably, between about 10⁸ to about 10¹⁰ particles are provided. Preferably, the preferred dose is 0.1% solids/ml. Therefore, for 500 nm beads, a preferred dose is approximately 4×10⁹ beads, for 50 nm beads, a preferred dose is approximately 4×10¹² beads. However, a dose that is effective in treating the particular condition to be treated is encompassed by the current invention.

The effectiveness of the nanoparticles described herein against the treatable diseases and conditions can be tested using a number of efficacy tests, including suitable animal models.

### Pharmaceutical Composition

One aspect of the present invention provides pharmaceutical compositions which comprise the subject nanoparticles, and optionally comprise a pharmaceutically acceptable carrier or excipient. Preferably, these compositions optionally further comprise one or more additional therapeutic agents. Alternatively, the subject particles of the current invention may be administered to a patient in need thereof in combination with the administration of one or more other therapeutic agents. For example, additional therapeutic agents for conjoint administration or inclusion in a pharmaceutical composition with a compound of this invention may be an approved anti-inflammatory agent, an immunotherapeutic agent, or a chemotherapeutic agent, or it may be any one of a number of agents undergoing approval in the Food and Drug Administration. It will also be appreciated that certain of the subject particles of the present invention can exist in free form for treatment, or where appropriate, as a pharmaceutically acceptable derivative thereof.

Preferably, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention.

It will also be appreciated that the nanoparticles and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects).

Preferably, the pharmaceutical compositions containing the particles of the present invention further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, anti-nausea medications and anti-sickness drugs.

The following examples are given to illustrate the present invention. It should be understood, however, that the invention is not to be limited to the specific conditions or details described in these examples.

### EXAMPLES

### Example 1 Preparation of Doxorubicin-Loaded PLGA Nanoparticles Having Transferrin on Surface

Approximately 40 mg of PLGA polymer and 5 mg doxorubicin are dissolved in 2 mL ethyl acetate. The resulting solution is added to a 15 ml glass vial containing 8 mL of 5% transferrin solution and approximately 0.7 ml ethyl acetate. The mixture is immediately homogenized using a probe sonicator at 90% amplitude for 30 seconds. The resulting emulsion is stirred magnetically for 3 hours to allow the solvent to evaporate and subsequently mixed with 200 ml DI water. The resulting mix is concentrated to approximately 10 mL using a TFF device with a molecular weight cutoff of 500 Kilo-Dalton. A fresh aliquot of 200 ml DI water is added to the concentrated nanoparticle suspension and concentrated again to approximately 10 ml. This wash-concentrating cycle is repeated for a third time and the resulting concentrated suspension is further washed by following the washing procedure below:
1) Divide the suspension into separate centrifuge tubes; centrifuge at 100,000 rcf for 10 min and decant the supernatant; analyze the supernatant to determine the concentration of free transferrin in the supernatant;
2) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge again at 100,000 rcf for 10 min and decant supernatant; analyze the supernatant to determine the concentration of free transferrin in the supernatant;
3) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge for the third time at 100,000 rcf for 10 min and decant supernatant; analyze the supernatant to determine the concentration of free transferrin in the supernatant;
4) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge for the fourth time at 100,000 rcf for 10 min and decant supernatant; analyze the supernatant to determine the concentration of free transferrin in the supernatant;
5) Add 1 ml of DI water to each centrifuge tube containing the nanoparticle pellet and resuspend the nanoparticles by vortexing and bath sonication; after nanoparticles are fully resuspended, combine the suspension in each tube into a new container;
6) Freeze and lyophilize the nanoparticle suspension. Such obtained nanoparticles have anti-tumor agent doxorubicin encapsulated inside and BBB-crossing agent transferrin tightly attached on the surface and have negligible amount of free transferrin.

### Example 2 Preparation of Curcumin-Loaded PLGA Nanoparticles Having ApoE on Surface

Approximately 40 mg of PLGA polymer and 5 mg curcumin are dissolved in 2 mL ethyl acetate. The resulting solution is added to a 15 ml glass vial containing 8 mL of 5% ApoE solution and approximately 0.7 ml ethyl acetate. The mixture is immediately homogenized using a probe sonicator at 90% amplitude for 30 seconds. The resulting emulsion is stirred magnetically for 3 hours to allow the solvent to evaporate and subsequently mixed with 200 ml DI water. The resulting mix is concentrated to approximately 10 mL using a TFF device with a molecular weight cutoff of 500 Kilo-Dalton. A fresh aliquot of 200 ml DI water is added to the concentrated nanoparticle suspension and concentrated again to approximately 10 ml. This wash-concentrating cycle is repeated for a third time and the resulting concentrated suspension is further washed by following the washing procedure below:
1) Divide the suspension into separate centrifuge tubes; centrifuge at 100,000 rcf for 10 min and decant the supernatant; analyze the supernatant to determine the concentration of free ApoE in the supernatant;
2) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge again at 100,000 rcf for 10 min and decant supernatant; analyze the supernatant to determine the concentration of free ApoE in the supernatant;
3) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge for the third time at 100,000 rcf for 10 min and decant supernatant; analyze the supernatant to determine the concentration of free ApoE in the supernatant;
4) Add 1 ml of DI water to each centrifuge tube containing the nanoparticle pellet and resuspend the nanoparticles by vortexing and bath sonication; after nanoparticles are fully resuspended, add another 2 ml DI water to the centrifuge tube; centrifuge for the fourth time at 100,000 rcf for 10 min and decant supernatant; analyze the supernatant to determine the concentration of free ApoE in the supernatant;
5) Add 1 ml of DI water to each centrifuge tube containing the nanoparticle pellet and resuspend the nanoparticles by vortexing and bath sonication; after nanoparticles are fully resuspended, combine the suspension in each tube into a new container;
6) Freeze and lyophilize the nanoparticle suspension. Such obtained nanoparticles have anti-tumor and anti-neurodegenerative agent curcumin encapsulated inside and BBB-crossing agent transferrin on the surface and contain negligible amount of free ApoE.

### Example 3. Preparation of Paclitaxel-Loaded PLGA Nanoparticles Having Both Transferrin and ApoE on Surface Via a Single-Emulsion Process

200 mg PLGA and 4 mg paclitaxel are dissolved in 4 mLethyl acetate to form a PLGA-paclitaxel solution. The PLGA-paclitaxel solution is mixed with 16 mL 2.5% polyvinyl alcohol solution containing 80 milligrams of transferrin and 80 milligrams of ApoE, and homogenized at 24,000 rpm for 1 minute using an IKA® DIGITAL ULTRA-TURRAX® T25 Homogenizer. The resulting emulsion is poured into a glass container and stirred magnetically at 400 rpm for 4 hours to allow the evaporation of the solvent. The paclitaxel loaded nanoparticles are then washed with distilled water until the concentration of free transferrin and ApoE in the supernatant is less than 0.01 mg/ml. Such obtained nanoparticles have an anti-tumor agent, paclitaxel, encapsulated inside the nanoparticles and two BBB-crossing agents, transferrin and ApoE tightly attached on the particle surface, and negligible amount of free transferrin and ApoE.

### Example 4. Preparation of Dalargin-Loaded PLGA Nanoparticles Having Transferrin on Surface Via a Double-Emulsion Process

200 mg PEG-PLGA is dissolved in 4 mL ethyl acetate to form a PLGA solution. A mix solution consisting of 35 ml 2% polyvinyl alcohol (PVA) solution (in water), 1.5 ml of ethyl acetate, and 80 milligrams of transferrin is prepared. 10 mg of dalargin is dissolved in 0.4 mL of an aqueous buffer to form a peptide solution. The peptide solution is mixed with the PLGA solution and the resulting mix is homogenized using a probe sonicator for 30 seconds. The resulting emulsion is mixed with the PVA/transferrin solution and homogenized at 24,000 rpm using a homogenizer for 1 minute. The resulting final emulsion is poured into a 250 mL glass flask and the solvent is removed by rotor evaporation at a vacuum of 50 mbar. The dalargin-loaded nanoparticles are washed three times with distilled water using a TFF device with a molecular weight cutoff of 500 Kilo-Dalton, and concentrated to approximately 10 ml. Such washed nanoparticle suspension is further washed by following the washing procedure below:
1) Transfer the nanoparticle suspension to centrifuge tubes; centrifuge at approximately 100,000 rcf for 10 min and decant the supernatant; analyze the supernatant to determine the concentration of free transferrin in the supernatant;
2) Add fresh DI water to the centrifuge tube containing the nanoparticle pellet and resuspend the nanoparticles by vortexing and bath sonication; after nanoparticles are fully resuspended, fill the centrifuge tube with fresh DI water; centrifuge again at approximately 100,000 rcf for 10 min and decant supernatant; analyze the supernatant to determine the concentration of free transferrin in the supernatant;
3) Repeat the above centrifugation-washing cycle until the concentration of free transferrin in the supernatant becomes less than 0.01 mg/ml;
4) Freeze and lyophilize the nanoparticle suspension. Such obtained nanoparticles have dalargin encapsulated inside and BBB-crossing agent transferrin tightly attached on the surface and contain negligible amount of free transferrin and can be used to for treating and managing pain.

### Example 5. Preparation of Urocortin-Loaded PLGA Nanoparticles Having Lactoferrin On Surface Via a Double-Emulsion Process

200 mg PEG-PLGA is dissolved in 4 mL ethyl acetate to form a PLGA solution. A mix solution consisting of 35 ml 2% polyvinyl alcohol (PVA) solution (in water), 1.5 ml of ethyl acetate, and 80 milligrams of lactoferrin is prepared. 1.0 mg of human urocortin is dissolved in 0.4 mL of an aqueous buffer to form a protein solution. The protein solution is mixed with the PLGA solution and the resulting mix is homogenized using a probe sonicator for 30 seconds. The resulting emulsion is mixed with the PVA/lactoferrin solution and homogenized at 24,000 rpm using a homogenizer for 1 minute. The resulting final emulsion is poured into a 250 mL glass flask and the solvent is removed by rotor evaporation at a vacuum of 50 mbar. The urocortin-loaded nanoparticles are washed three times with distilled water using a TFF device with a molecular weight cutoff of 500 Kilo-Dalton, and concentrated to approximately 10 ml. Such washed nanoparticle suspension is further washed by following the washing procedure below:
1) Transfer the nanoparticle suspension to centrifuge tubes; centrifuge at approximately 100,000 rcf for 10 min and decant the supernatant; analyze the supernatant to determine the concentration of free lactoferrin in the supernatant;
2) Add fresh DI water to the centrifuge tube containing the nanoparticle pellet and resuspend the nanoparticles by vortexing and bath sonication; after nanoparticles are fully resuspended, fill the centrifuge tube with fresh DI water; centrifuge again at approximately 100,000 rcf for 10 min and decant supernatant; analyze the supernatant to determine the concentration of free lactoferrin in the supernatant;
3) Repeat the above centrifugation-washing cycle until the concentration of free lactoferrin in the supernatant becomes less than 0.01 mg/ml;
4) Freeze and lyophilize the nanoparticle suspension. Such obtained nanoparticles have urocortin encapsulated inside and BBB-crossing agent lactoferrin tightly attached on the surface and contain negligible amount of free lactoferrin and can be used to for treating neurodegenerative diseases such as Parkinson's Disease and Alzheimer's Disease.

### Example 6. Preparation of a Fluorescently Labeled Oligonucleotide

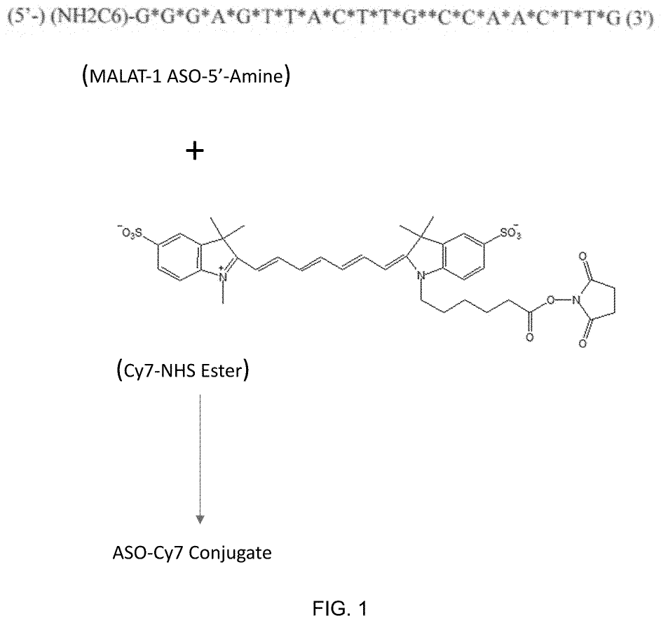

An antisense oligonucleotide (ASO) targeting a non-coding nuclear RNA, metastasis-associated lung adenocarcinoma transcript 1 (MALAT1) with a primary amine group added at the 5′-position (see FIG. 1) was obtained from Boston Open Labs, Cambridge, Mass. Such MALAT1-ASO-5′-amine was reacted with equal molar quantity of a Cy7 near-IR fluorescent dye functionalized with NHS ester. The resulting reaction product is referred to as ASO-Cy7 conjugate.

### Example 7. Preparation of Nanoparticles Loaded with the ASO-Cy7 Conjugate and Having Transferrin Presented on the Surface

Approximately 10 mg of the ASO-Cy7 conjugate prepared in Example 6 was dissolved in 0.1 mL distilled water to form an ASO solution. 50 mg of poly (lactide-co-glycolide) (PLGA, ester end-capped) was dissolved in 0.8 mL ethyl acetate to form a polymer solution. 12.75 mg of ethyl lauroyl arginate (ELA) was dissolved in 0.2 mL benzyl alcohol to form an ELA solution. The polymer solution and the ELA solution were mixed in an 8-mL glass vial followed by the addition of the ASO solution. The resulting mixture in the 8-mL vial was probe-sonicated at 90% amplitude for 30 seconds to result in a first emulsion, which was transferred into a 15-mL glass vial containing 5 mL of an aqueous solution consisting of 0.5% poly(vinyl alcohol) (PVA, 89% hydrolyzed), 0.2% Brij-S100-PA-SG (Brij-S100), and 0.2% transferrin saturated with appropriate amount of ethyl acetate. The entire mixture was immediately probe-sonicated at 90% amplitude for 60 seconds to result in a second emulsion, which was transferred to a 30 mL beaker and stirred magnetically in a chemical fume hood for 2 hours. Once the particles were formed and hardened, the suspension was washed with 50 mL phosphate buffer saline followed by 50 mL distilled water twice using tangential flow filtration. After purification, the suspension was concentrated to 2 mL. The nanoparticles obtained were found to have an average particle size of 88.03 nm, a loading of the ASO-Cy7 of 3.6%, and a surface transferrin loading of 13.5%.

### Example 8. In Vivo Animal Studies on the Blood-Brain Barrier (BBB) Crossing by the ASO-Cy7 Loaded Nanoparticles Having Transferrin on Surface

1) Dosing:

ASO-Cy7 conjugate was dissolved in a buffer solution and administered to 5 CD-1 female mice via the lateral tail vein; ASO-Cy7 NPs were suspended in a PBS buffer and dosed similarly. Dosage was 1.5 mg/kg for the free ASO-Cy7 group and 150 mg/kg (6 mg/kg Cy7-ASO) for the nanoparticle group.

2) In Vivo Imaging:
The in vivo imaging was performed at 15 min, 1 hr, and 4 hr post injection, respectively.

3) Ex Vivo Imaging and Biodistribution
At 4 hr, collect blood and euthanize mice, perfuse carcass and acquire ex vivo image of brain, plasma and major organs (lungs, heart, liver, spleen, and kidneys).

4) Fluorescent Assay
After ex vivo imaging, the brain was cut at the midline. One brain hemisphere was positioned with the flat midline down in the mold for OCT embedding. The remaining hemisphere was homogenized into single cell suspension and Cy7 concentration was determined via a 96-well plate reader.

5) Histology
Histology study was conducted on the brain tissue. Nuclei was stained with DAPI and neuronal cells stained with FITC. Sample slides were imaged on a fluorescent microscope and a confocal microscope, respectively.

While this invention has been particularly shown and described with references to preferred embodiments thereof, it will be understood by those skilled in the art that various changes in form and details may be made therein without departing from the scope of the invention encompassed by the appended claims.

It should be understood that any preferred features of the invention described herein can be combined with any other preferred features, including preferred features described only under one aspect of the invention, and preferred features described only in the examples. Throughout the specification, any and all references to a publicly available document, including any U.S. patent or patent application publication, are specifically incorporated by reference.

What is claimed is:

1. Particles comprising a biodegradable polymer and a blood brain barrier (BBB)-crossing agent intertwined on the surface thereof, wherein the BBB-crossing agent is a peptide or protein having an affinity for a component of the blood brain barrier.

2. The particles of claim 1, wherein the particles are microparticles or nanoparticles.

3. The particles of claim 1, wherein the biodegradable polymer is polylactide (PLA), poly(lactide-co-glycolide) (PLGA), copolymers of ethylene glycol and lactide/glycolide (PEG-PLGA), copolymers of ethylene glycol and lactide (PEG-PLA), copolymers of ethylene glycol and glycolide (PEG-PGA), poly(ethylene glycol) (PEG), polycaprolactone (PCL), polyanhydrides (PANH), poly(ortho esters), polycyanoacrylates, poly(hydroxyalkanoate)s (PHAs), poly(sebasic acid), polyphosphazenes, polyphosphoesters, modified poly(saccharide)s, poly(amino esters), dendrimers, chitosan, gelatin, human serum albumin (HSA), hyluronic acid, dextran, mixtures and copolymers thereof, preferably PLGA or poly(n-butyl cyanoacrylate) (PBCA).

4. The particles of claim 1, wherein the biodegradable polymer is PLGA or PBCA.

5. The particles of claim 4, wherein the BBB-crossing agent and biodegradable polymer form an interpenetrating network.

6. The particles of claim 4, wherein the BBB-crossing agent is selected from the group consisting of transferrin, lactoferrin, insulin, low-density lipoprotein (LDL), apolipoproteins, ApoE, cell-penetrating peptides, penetratin, anti-transferrin receptor (TfR) antibody, ligands for transport proteins, GLUT1 and ACST2 or a BBB-binding fragments of any of the above.

7. The particles of claim 6, wherein the BBB-crossing agent is transferrin.

8. The particles of claim 1, further comprising an active agent.

9. The particles of claim 8, wherein the active agent is encapsulated inside the particles.

10. The particles of claim 8, wherein the active agent is a selected from the group containing small molecule compound, peptide, protein, oligonucleotide, RNA and DNA.

11. The particles of claim 10, wherein the active agent is an oligonucleotide.

12. The particles of claim 11, where in the active agent is an antisense oligonucleotide.

13. The particles of claim 1, further comprising a targeting agent bound to the surface of the particles.

14. A method for the preparation of microparticles or nanoparticles comprising: (1) dissolving a biodegradable polymer (and optionally an active agent, such as a pharmaceutical ingredient (API), or a poorly water soluble compound) in a first solvent to form a polymer solution; (2) emulsifying the polymer solution in a solution of a second solvent to form an emulsion, wherein the first solvent is not miscible or partially miscible with the second solvent, and wherein the solution of the second solvent comprises a BBB-crossing agent and a surfactant; and, (3) removing the first solvent to form said microparticles or nanoparticles having the BBB-crossing agent intertwined on the surface thereof.

15. The method of claim 14 comprising: (1) dissolving biodegradable polymer (and optionally an active agent, an API, or a poorly water soluble compound) in a first solvent to form a polymer solution; (2) adding a second solvent to the polymer solution to form a mixture, wherein the first solvent is not miscible or partially miscible with the second solvent, and wherein the first solution of the second solvent (optionally comprises an active agent which may be the same or different); (3) emulsifying the mixture to form a first emulsion; (4) emulsifying the first emulsion in a second solution of the second solvent to form a second emulsion, wherein the second solution of the second solvent comprises a BBB-crossing agent, and optionally further comprises a surfactant; and, (5) removing the first solvent to form microparticles or nanoparticles having the BBB-crossing agent intertwined on the surface thereof.

16. The method of claim 15, further comprising washing said microparticles or nanoparticles, and/or concentrating said microparticles or nanoparticles to a desired volume.

17. The method of claim 14, wherein the biodegradable polymer is polylactide (PLA), poly(lactide-co-glycolide) (PLGA), copolymers of ethylene glycol and lactide/glycolide (PEG-PLGA), copolymers of ethylene glycol and lactide (PEG-PLA), copolymers of ethylene glycol and glycolide (PEG-PGA), poly(ethylene glycol) (PEG), polycaprolactone (PCL), polyanhydrides (PANH), poly(ortho esters), polycyanoacrylates, poly(hydroxyalkanoate)s (PHAs), poly(sebasic acid), polyphosphazenes, polyphosphoesters, modified poly(saccharide)s, poly(amino esters), dendrimers, chitosan, gelatin, human serum albumin (HSA), hyluronic acid, dextran, mixtures and copolymers thereof, preferably PLGA or poly(n-butyl cyanoacrylate) (PBCA).

18. The method of claim 17, wherein the biodegradable polymer is PLGA or PBCA.

19. The method of claim 18, wherein the BBB-crossing agent and biodegradable polymer form an interpenetrating network.

20. The method of claim 19, wherein the BBB-crossing agent is selected from the group consisting of transferrin, lactoferrin, insulin, low-density lipoprotein (LDL), apolipoproteins, ApoE, cell-penetrating peptides, penetratin, anti-transferrin receptor (TfR) antibody, ligands for transport proteins, GLUT1 and ACST2 or a BBB-binding fragments of any of the above.

21. The method of claim 14, wherein the active agent is a selected from the group containing small molecule compound, peptide, protein, oligonucleotide, RNA and DNA.

22. The method of claim 21, wherein the active agent is an oligonucleotide.

23. The method of claim 14, wherein the first solvent is methylene chloride, ethyl acetate, or chloroform.

24. The method of claim 14, wherein the solution of the second solvent is aqueous and preferably comprises a surfactant comprising organic or inorganic pharmaceutical excipients; various polymers; oligomers; natural products; nonionic, cationic, zwitterionic, or ionic surfactants; and mixtures thereof, preferably the surfactant is selected form polyvinyl alcohol (PVA), polyvinylpyrrolidone (PVP), a Tween series surfactant, Pluronic series, Poloxamer series, or Triton X-100 or a salt, derivative, copolymer, or mixture thereof.

25. The method of claim 14, wherein the emulsifying step comprises homogenization, mechanical stirring, and/or microfluidization.

26. The method of claim 14, wherein the first solvent is removed through solvent exchange and/or evaporation.
